(19) United States
(12) Patent Application Publication
(10) Pub. No.: US 2023/0277044 A1
(43) Pub. Date: Sep. 7, 2023

(54) OTOSCOPE SEAL DEVICES AND METHODS

(71) Applicant: OtoNexus Medical Technologies, Inc., Bellevue, WA (US)

(72) Inventors: Mark A. Moehring, Seattle, WA (US); Andrew S. Kessler, Seattle, WA (US); Bethany Johnson, Kirkland, WA (US); Charlie Corredor, Seattle, WA (US); Caitlin Cameron, Mercer Island, WA (US); Chad Jason MacDonald, Bothell, WA (US)

(73) Assignee: OtoNexus Medical Technologies, Inc., Bellevue, WA (US)

(21) Appl. No.: 18/065,103

(22) Filed: Dec. 13, 2022

Related U.S. Application Data
(63) Continuation of application No. PCT/US2021/040370, filed on Jul. 2, 2021.
(60) Provisional application No. 63/048,312, filed on Jul. 6, 2020.

(51) Int. Cl. A61B 1/00; A61B 1/227; A61B 8/12; A61B 5/12; A61B 1/32

(52) U.S. Cl. CPC A61B 1/00137; A61B 1/2275; A61B 8/12; A61B 5/123; A61B 1/0011; A61B 1/32

(57) ABSTRACT

A speculum of an otoscope may include a housing and a seal. The seal may include a surface sized and shaped to interface with an external auditory meatus. In some examples, the seal comprises one or more compressible members deformable to interface with the external auditory meatus. The seal may be configured to retain a volume of fluid within a lumen of the speculum in response to one or more of a negative pressure change or a positive pressure change.

### CROSS-REFERENCE

This application is a continuation of PCT Application No. PCT/US21/40370, filed Jul. 2, 2021, which claims the benefit of U.S. Provisional Application No. 63/048,312, filed Jul. 6, 2020, which application is incorporated herein by reference in its entirety for all purposes.

### BACKGROUND

Pneumatic otoscopy is a medical examination that allows determination of the mobility of a tympanic membrane of a patient in response to pressure changes (e.g., airflow). A healthy tympanic membrane moves in response to pressure. Immobility may be due to fluid in the middle ear. Otitis media with effusion is characterized by the presence of fluid adjacent the tympanic membrane. Accordingly, establishing a diagnosis of otitis media with effusion may be aided by pneumatic otoscopy.

Pneumatic otoscopy generally comprises insertion of a speculum into the ear canal to create a pressure seal. The proximal end of the speculum tip is attached to a head comprising a lens, a light source, and a pressure source (e.g., a rubber bulb). The clinician gently squeezes and releases the bulb to induce movement in the tympanic membrane. The clinician then observes the presence or absence of membrane movement to infer the presence of fluid behind the membrane.

### SUMMARY

Recognized herein is a need to improve the seal between a speculum and an external ear canal. A seal prevents air leakage between the speculum and ear canal wall. An improper seal can lead to a lack of sufficient pressure to move the tympanic membrane and result in a false diagnosis of middle ear effusion. An adequate seal may be difficult to attain for a wide variety of ear canal shapes and a wide variety of patient ages. Apart from the difficultly of obtaining an adequate seal, no contraindications exist for pneumatic otoscopy. Devices and methods disclosed herein are generally directed to improved speculum seals for otoscopy measurements.

In an aspect, the present disclosure provides a speculum operable to be disposed within an ear of a subject. The speculum may comprise: a housing comprising a lumen configured to allow a pressure challenge wave to be propagated therethrough, wherein the housing comprises a distal end sized and shaped to be disposed within an external auditory meatus of a subject; and a seal comprising a surface sized and shaped to interface with the external auditory meatus, wherein the seal comprises one or more compressible members deformable to interface with the external auditory meatus, and wherein the seal is configured to retain a volume of fluid within the lumen in response to one or more of a negative pressure change or a positive pressure change.

In some embodiments, the seal is constructed from a material comprising a Shore durometer between about 40 to about 80 Shore-alpha. In some embodiments, the material is polydimethylsiloxane. In some embodiments, the one or more compressible members comprises a plurality of compressible members. In some embodiments, the plurality of compressible members comprises a plurality of flanges. In some embodiments, the flanges comprise monotonically decreasing diameters. In some embodiments, a flange of the plurality of flanges comprises a gel within a volume radially near the housing. In some embodiments, the volume is disposed on a proximal side of the flange. In some embodiments, the volume is disposed on a distal side of the flange. In some embodiments, the seal is releasably couplable to the housing. In some embodiments, the seal comprises one or more retention features which secure the seal to the housing. In some embodiments, the retention features comprise a tension fit.

In some embodiments, the seal provides a barrier between the lumen of the housing and an exterior of the housing not engaged with or within the external auditory meatus. In some embodiments, the negative pressure change or the positive pressure change comprises a magnitude of greater than about 30 mm H₂O. In some embodiments, the negative pressure change or the positive pressure change comprises a magnitude of about 30 mm H₂O to about 100 mm H₂O.

In some embodiments, the speculum is releasably couplable to an otoscope. In some embodiments, the otoscope is a pneumatic otoscope. In some embodiments, the pneumatic otoscope comprises an ultrasound transducer. In some embodiments, the pneumatic otoscope is configured to generate the negative pressure change or the positive pressure change. In some embodiments, the pneumatic otoscope comprises an indication that the pressure challenge wave within the lumen of the speculum is substantially retained within the lumen.

In some embodiments, a most distal of the one or more compressible members comprises a setback of greater than about 2 mm. In some embodiments, the setback is about 5 mm to about 7 mm. In some embodiments, a most distal of the one or more compressible members is configured to allow the speculum to enter at least about 5 mm within the external auditory meatus.

In another aspect, the present disclosure provides seal for use with a speculum of an otoscope. The seal may comprise: a surface sized and shaped to interface with an external auditory meatus, wherein the seal comprises one or more compressible members deformable to interface with the external auditory meatus, and wherein the seal is configured to retain a volume of fluid within a lumen of the speculum in response to one or more of a negative pressure change or a positive pressure change.

In some embodiments, the seal is constructed from a material comprising a Shore durometer between about 40 to about 80 Shore-alpha. In some embodiments, the material is polydimethylsiloxane. In some embodiments, the one or more compressible members comprises a plurality of compressible members. In some embodiments, the plurality of compressible members comprises a plurality of flanges. In some embodiments, the flanges comprise monotonically decreasing diameters. In some embodiments, a flange of the plurality of flanges comprises a gel within a volume radially near the housing. In some embodiments, the volume is disposed on a proximal side of the flange. In some embodiments, the volume is disposed on a distal side of the flange. In some embodiments, the seal is releasably couplable to a housing. In some embodiments, the seal comprises one or more retention features which secure the seal to the housing. In some embodiments, the retention features comprise a tension fit.

In some embodiments, the seal provides a barrier between the lumen of the housing and an exterior of the housing not engaged with or within the external auditory meatus. In some embodiments, the negative pressure change or the positive pressure change comprises a magnitude of greater than about 30 mm H₂O. In some embodiments, the negative pressure change or the positive pressure change comprises a magnitude of about 30 mm H₂O to about 100 mm H₂O.

In some embodiments, the seal is releasably couplable to a speculum of an otoscope. In some embodiments, the otoscope is a pneumatic otoscope. In some embodiments, the pneumatic otoscope comprises an ultrasound transducer. In some embodiments, the pneumatic otoscope is configured to generate the negative pressure change or the positive pressure change. In some embodiments, the pneumatic otoscope comprises an indication that the pressure challenge wave within the lumen of the speculum is substantially retained within the lumen.

In some embodiments, a most distal of the one or more compressible members comprises a setback of greater than about 2 mm. In some embodiments, the setback is about 5 mm to about 7 mm. In some embodiments, a most distal of the one or more compressible members is configured to allow the speculum to enter at least about 5 mm within the external auditory meatus.

In another aspect, the present disclosure provides a device for interfacing with an ear of a subject. The device may comprise: a speculum comprising a distal end sized and shaped to be disposed within an external auditory meatus of a subject, wherein the speculum comprises a lumen; a pressure generator, wherein the pressure generator creates a pressure challenge wave which propagates through the lumen, wherein the pressure challenge wave comprises one or more of a negative pressure change or a positive pressure change; a seal comprising a surface sized and shaped to interface with an external auditory meatus, wherein the seal comprises one or more compressible members deformable to interface with the external auditory meatus; and a pressure gauge, wherein the pressure gauge comprises an indication that the pressure challenge wave within the lumen of the speculum is substantially retained within the lumen.

In some embodiments, the seal is constructed from a material comprising a Shore durometer between about 40 to about 80 Shore-alpha. In some embodiments, the material is polydimethylsiloxane. In some embodiments, the one or more compressible members comprises a plurality of compressible members. In some embodiments, the plurality of compressible members comprises a plurality of flanges. In some embodiments, the flanges comprise monotonically decreasing diameters. In some embodiments, a flange of the plurality of flanges comprises a gel within a volume radially near the housing. In some embodiments, the volume is disposed on a proximal side of the flange. In some embodiments, the volume is disposed on a distal side of the flange. In some embodiments, the seal is releasably couplable to a housing. In some embodiments, the seal comprises one or more retention features which secure the seal to the housing. In some embodiments, the retention features comprise a tension fit.

In some embodiments, the seal provides a barrier between the lumen of the housing and an exterior of the housing not engaged with or within the external auditory meatus. In some embodiments, the negative pressure change or the positive pressure change comprises a magnitude of greater than about 30 mm H₂O. In some embodiments, the negative pressure change or the positive pressure change comprises a magnitude of about 30 mm H₂O to about 100 mm H₂O.

In some embodiments, the seal is releasably couplable to the speculum and wherein the speculum is coupled to an otoscope. In some embodiments, the otoscope is a pneumatic otoscope. In some embodiments, the pneumatic otoscope comprises an ultrasound transducer.

In some embodiments, a most distal of the one or more compressible members comprises a setback of greater than about 2 mm. In some embodiments, the setback is about 5 mm to about 7 mm. In some embodiments, a most distal of the one or more compressible members is configured to allow the speculum to enter at least about 5 mm within the external auditory meatus.

In another aspect, the present disclosure provides a method for characterizing a tympanic membrane of a subject. The method may comprise: providing a pneumatic otoscope comprising a speculum coupled thereto, wherein the speculum comprises a lumen configured to allow a pressure challenge wave to be propagated therethrough, wherein the speculum comprises a distal end sized and shaped to be disposed within an external auditory meatus of a subject; providing a seal comprising a surface sized and shaped to interface with the external auditory meatus, wherein the seal comprises one or more compressible members deformable to interface with the external auditory meatus; and generating a negative pressure change or a positive pressure change, wherein the seal substantially retains a volume of fluid within the lumen in response to the negative pressure change or the positive pressure change.

In some embodiments, the speculum is the speculum of any aspect or embodiment disclosed herein. In some embodiments, the seal is the seal of any aspect or embodiment disclosed herein. In some embodiments, the pneumatic otoscope comprises the device of any aspect or embodiment disclosed herein.

In another aspect, the present disclosure provides a method of manufacturing a speculum tip. The method may comprise: providing a speculum comprising a lumen configured to allow a pressure challenge wave to be propagated therethrough, wherein the speculum comprises a distal end sized and shaped to be disposed within an external auditory meatus of a subject; attaching a seal comprising a surface sized and shaped to interface with the external auditory meatus, wherein the seal comprises one or more compressible members deformable to interface with the external auditory meatus, and wherein the seal is configured to retain a volume of fluid within the lumen in response to one or more of a negative pressure change or a positive pressure change.

In some embodiments, the speculum is the speculum of any aspect or embodiment disclosed herein. In some embodiments, the seal is the seal of any aspect or embodiment disclosed herein. In some embodiments, the method further comprises providing the device of any aspect or embodiment disclosed herein.

In another aspect, the present disclosure provides a computer implemented method of determining a geometry of a speculum tip. The method may comprise: providing a database of ear geometries; providing a database of speculum geometries; using a model of the insertion of a plurality of speculum geometries within the database of speculum geometries into a plurality of ear geometries within the database of ear geometries to determine a speculum diameter which provides a seal and a remaining open cross sectional area; and selecting a speculum geometry from the plurality of speculum geometries based on the speculum diameter and the open cross sectional area.

In some embodiments, selecting a speculum geometry comprises selecting a geometry of one or more compressible members of a speculum seal. In some embodiments, the database of ear geometries is based on computed tomography scans from a plurality of subjects. In some embodiments, the method further comprises providing an indication to a user to provide a speculum comprising the speculum geometry. In some embodiments, the speculum comprises the speculum of any aspect or embodiment disclosed herein.

Another aspect of the present disclosure provides a system comprising one or more computer processors and computer memory coupled thereto. The computer memory comprises machine executable code that, upon execution by the one or more computer processors, implements any of the methods above or elsewhere herein, such as for example, the method of determining a geometry of a speculum tip.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a side view of an example of a speculum S within an external auditory meatus EAM.

FIGS. 2A-2C shows the TM deflection and measured backing pressure in response to a series of positive and negative step function excitations applied to a healthy example TM.

FIG. 3A depicts an anatomical model of an ear based on computerized tomography (CT) data of a right ear of a 0.6 year old female.

FIG. 3B depicts a cross section of ear model of FIG. 3A at a point within the EAM.

FIGS. 4A-4E illustrate an example of a seal with a single compressible member in the form of a torus.

FIGS. 5A-5E illustrate an example of a seal with a single compressible member in the form of a cone.

FIGS. 6A and 6B illustrate an example of a seal with a single compressible member in the form of a sphere.

FIGS. 7A and 7B illustrate an example of a seal with a single compressible member in the form of an upturned cone.

FIGS. 8A and 8B illustrate an example of a seal with a single compressible member in the form of an upturned cone with a compressible gel.

FIGS. 9A and 9B illustrate an example of a seal with a single compressible member in the form of a hollow cone.

FIGS. 10A-10C illustrate an example of a seal with a single compressible member in the form of a bulb.

FIGS. 11A-11D illustrate an example of a seal with a collar and a single compressible member in the form of a convex cone.

FIGS. 12A-12D illustrate an example of a seal with a collar and a single compressible member in the form of a concave cone.

FIGS. 13A-13G illustrate an example of a seal with three flanges of varying diameter.

FIGS. 14A-14F illustrate an example of a seal with four flanges of varying diameter.

FIGS. 15A-15C illustrate an example of a seal with three flanges each comprising a cone shaped profile.

FIGS. 16A and 16B illustrate an example of a seal with four flanges each comprising a cone shaped profile.

FIGS. 17A and 17B illustrate an example of a seal with 4 flanges in the form of flat tiers.

FIGS. 18A-18C illustrate an example of a seal with four flanges comprising space for a gel in a proximal volume.

FIGS. 19A and 19B illustrate an example of a seal with 9 flanges in the form of flat tiers.

FIGS. 20A-20E illustrate an example of a seal formed with 4 flanges supported by perpendicular struts.

FIG. 21A is a plot of an ear insertion model for an example speculum tip inside of an example EAM geometry.

FIG. 21B is a series of histograms for a plurality of subjects indicating: a length of the EAM, for example, the depth to the tympanic membrane; the modeled insertion depth; and the conflict point.

FIG. 22A is a plot including a series of EAM profiles aligned by conflict point with an example speculum tip.

FIG. 22B illustrates FIG. 22A showing only data proximal of the conflict point as well as the profile at which 50% and 80% of EAMs are sealed.

### DETAILED DESCRIPTION

The devices, otoscopes, specula, and methods of use and manufacture thereof as disclosed herein may address issues related to measurement of a movement of a tympanic membrane in response to a pneumatic challenge. For example, the present disclosure may improve upon a seal between an external auditory meatus (as referred to herein as the external ear canal or external auditory canal) and a speculum of an otoscope. The devices, otoscopes, specula, and methods of use and manufacture thereof as disclosed herein may address extant difficulties in the field with respect to taking accurate pneumatic otoscopy measurements, such as, for example, excess leakage of pressure generated by the pneumatic challenge with may result in less accurate measurements. In some cases, present disclosure addresses issues in the field of otoscopy.

For example, surface characterization using an analysis of reflected ultrasound in the presence of a pneumatic excitation may be improved if a more consistent and/or more predictable pneumatic challenge is delivered to a tympanic membrane. Because a leak between a speculum and an external auditory meatus may result in pressure being directed out of an ear rather than at a tympanic membrane, an improved seal may result in improved measurements. In some cases, the present disclosure provides improved specula, seals for specula, otoscopes, and methods of use and manufacture thereof.

The devices, otoscopes, specula, and methods of use and manufacture thereof as disclosed herein provide at least some of the following advantages: creating a static or constant fluid (air) between the external ear canal and the speculum tip, when the speculum is in position for observing the tympanic membrane; limiting penetration of air between the ear canal and the speculum tip; creating a seal that limits invasion of the external ear canal; creating a seal that predominately acts on the external auditory meatus; creating a seal with a low friction coefficient for insertion and removal during examination; limiting patient discomfort; limiting user (e.g. clinician) discomfort; increasing a surface area of contact where force is applied, thereby minimizing contact pressure; allowing the operator to manipulate the speculum tip off the center axis; allowing manipulation of the speculum tip off the center axis of the ear canal; allowing the manipulation of the speculum tip in a radial direction, to allow for tympanic membrane imaging purposes; allowing for pitch, yaw, and roll of the speculum tip, e.g., in the process of visualizing the cone of light; creating a seal which is biocompatible; providing a seal which does not impede observation of speculum tip insertion to the external ear canal; and providing a seal which is easy to manufacture.

The devices, otoscopes, specula, and methods of use and manufacture thereof as disclosed herein may be used in combination with for example devices and methods to characterize a ductile membrane, surface, and sub-surface properties such as those described in commonly owned U.S. Pat. No. 7,771,356 and U.S. Patent Publication Nos. 2019/0365292, 2018/0310917, and 2017/0014053, each of which is incorporated by reference in their entireties. The devices, otoscopes, specula, and methods of use and manufacture thereof as disclosed herein may be used in combination with for example devices and methods using optical coherence tomography (OCT), as disclosed in commonly assigned U.S. Patent Publication No. 2019/0200873 and U.S. Patent Publication No. 2017/0360302, each of which is incorporated herein by reference in its entirety.

The devices, otoscopes, specula, and methods of use and manufacture thereof as disclosed herein may be used in combination with for example devices and methods to transmit optical illumination such as those described in commonly owned U.S. Patent Publication 2020/0107813, which is incorporated by reference in its entirety.

The following references may be of interest: Schwartz, Richard H., and Daniel M. Schwartz, “Acute otitis media: Diagnosis and drug therapy,” Drugs 19.2 (1980): 107-118; Cavanaugh, Robert M, “Pneumatic otoscopy in healthy full-term infants,” Pediatrics 79.4 (1987): 520-523; Jones, Woodson S., and Phillip H. Kaleida, “How helpful is pneumatic otoscopy in improving diagnostic accuracy?” Pediatrics 112.3 (2003): 510-513; Kaleida, Phillip H., and Sylvan E. Stool, “Assessment of otoscopists' accuracy regarding middle-ear effusion: otoscopic validation,” American Journal of Diseases of Children 146.4 (1992): 433-435; Morris, Elizabeth, et al., “Development and validation of a novel ear simulator to teach pneumatic otoscopy,” Simulation in Healthcare 7.1 (2012): 22-26; U.S. Pat. No. 5,919,130; and U.S. Patent Publication No. 2019/0142258, each of which is incorporated by reference herein for all purposes.

The devices, otoscopes, specula, and methods of use and manufacture thereof as disclosed herein may be used to characterize a number of biological tissues to provide a variety of diagnostic information. A biological tissue may comprise a patient organ. A speculum may be disposed within a bodily cavity to characterize a patient tissue. A patient organ or bodily cavity may comprise for example: a muscle, a tendon, a ligament, a mouth, a tongue, a pharynx, an esophagus, a stomach, an intestine, an anus, a liver, a gallbladder, a pancreas, a nose, a larynx, a trachea, lungs, a kidneys, a bladder, a urethra, a uterus, a vagina, an ovary, a testicle, a prostate, a heart, an artery, a vein, a spleen, a gland, a brain, a spinal cord, a nerve, etc, to name a few.

The devices, otoscopes, specula, and methods of use and manufacture thereof as disclosed herein may be used to characterize a tympanic membrane. For example, a membrane may be characterized to determine a condition of an ear, such as acute otitis media (AOM). A characterization that an ear exhibits AOM may include detection of the presence of effusion and characterization of the type of effusion as one of serous, mucoid, purulent, or combinations of these. In AOM, the middle ear effusion (MEE) may be induced by infective agents and may be thin or serous with viral infection and thicker and purulent with bacterial infection. Accordingly, determining various properties of a fluid adjacent a tympanic membrane may provide information which may be used to characterize a membrane.

Reference will now be made in detail to various embodiments, examples of which are illustrated in the accompanying drawings. In the following detailed description, numerous specific details are set forth in order to provide a thorough understanding of the present disclosure and the described embodiments. However, the embodiments of the present disclosure are optionally practiced without these specific details. In other instances, well-known methods, procedures, components, and circuits have not been described in detail so as not to unnecessarily obscure aspects of the embodiments. In the drawings, like reference numbers designate like or similar steps or components.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the terms “subject” and “patient” are used interchangeably. As used herein, the terms “subject” and “subjects” refers to an animal (e.g., birds, reptiles, and mammals), a mammal including a primate (e.g., a monkey, chimpanzee, and a human) and a non-primate (e.g., a camel, donkey, zebra, cow, pig, horse, cat, dog, rat, and mouse). In certain embodiments, the mammal is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old, or 95 to 100 years old.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range. In certain embodiments, the term “about” or “approximately” means within 40.0 mm, 30.0 mm, 20.0 mm, 10.0 mm 5.0 mm 1.0 mm, 0.9 mm, 0.8 mm, 0.7 mm, 0.6 mm, 0.5 mm, 0.4 mm, 0.3 mm, 0.2 mm or 0.1 mm of a given value or range.

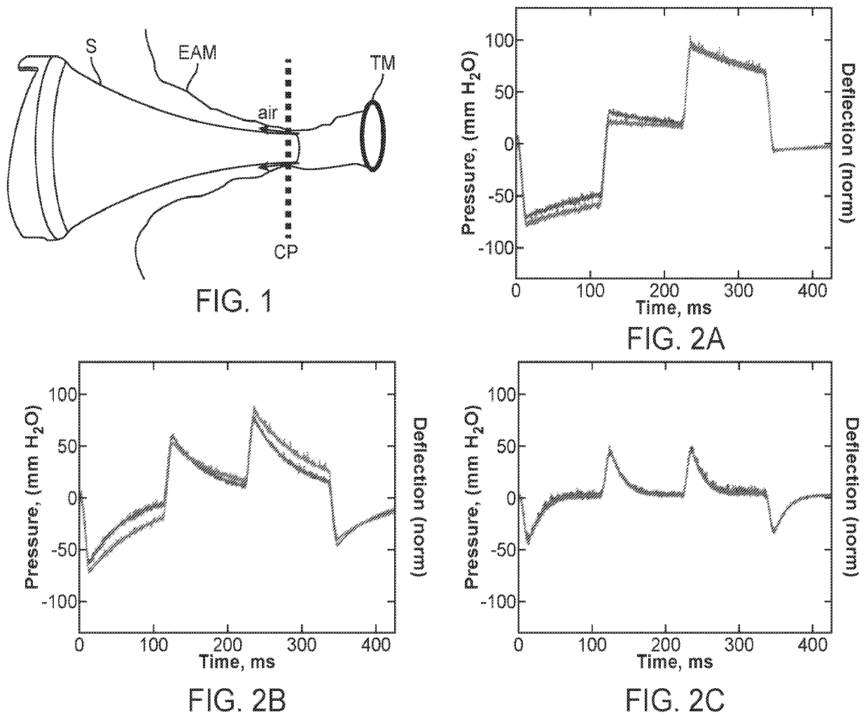

FIG. 1 is a side view of an example of a speculum S within an external auditory meatus EAM. As shown, a distal end of the speculum tip may be placed within external auditory meatus EAM. The EAM may be a pathway from the outer ear to the middle ear from the pinna to the tympanic membrane TM. The EAM may vary in length from person to person; however, the EAM may generally be longer and larger in diameter in adults than in children. For example, an adult EAM may be about 2.5 centimeters (cm) long, and at birth the EAM may be about 14 to 15 millimeters (mm) long. The EAM may comprise a bony portion and a cartilaginous portion. The bony portion of the ear canal may extend in length with age from a ring in a newborn to about ⅔ of the length of the canal in an adult. The EAM may not generally be straight. Further, the EAM may not generally be round in cross-section.

Contact between a speculum tip and the TM may not be advantageous. In some cases, it may be possible to damage the TM by physical contact with speculum. In some cases, it may be possible to damage the TM with a pneumatic challenge directed a too short a distance or too great a pressure toward the TM. The devices, otoscopes, specula, and methods of use and manufacture thereof as disclosed herein may avoid or prevent injury to the TM.

FIG. 1 shows a dotted line at the contact point CP between a speculum tip and the EAM. In some cases, it may be useful to provide a speculum tip which cannot physically reach a depth to damage an ear drum. However, a measurement may require a speculum tip to reach a sufficient distance to take a measurement. The CP may be used to characterize when a diameter of a speculum is too large to reach a sufficient depth.

FIG. 1 shows a gas (e.g. air) flowing proximally out from the EAM. A gas flow (e.g., a leak) may be caused by a shape of a speculum tip not adequately fitting an interior of an EAM. An inadequate fit may be caused by mismatching in the shape of an EAM, which may not generally be round, and the speculum tip, which often is. Further, the speculum tip may be too small to ever form a contact point CP.

The devices, otoscopes, specula, and methods of use and manufacture thereof as disclosed herein may be used in conjunction with various devices, systems, and methods obtaining information regarding the motion of a tympanic membrane. In some cases, the devices, otoscopes, specula, and methods of use and manufacture thereof as disclosed herein may be used in conjunction with devices, systems and methods of using ultrasound echo signals as disclosed in the incorporated references may be used to generate one or more parameters related to a dynamic property of the tympanic membrane. For example, a system for measuring ultrasound echo signal may induce motion of the tympanic membrane by applying a systematic pressure pulse and then extracting Doppler shift signals from ultrasound waves to analyze displacement of the TM and/or categorize viscosity of ear effusion.

FIGS. 2A-2C show examples of pneumatic otoscopy data taken with various seal qualities, in accordance with embodiments. FIGS. 2A-2C show pneumatic otoscopy data taken in response to a pneumatic excitation. A pneumatic excitation may comprise a pressure excitation, such as an air puff. A pneumatic excitation may change a response of a membrane to ultrasound excitation. For example, a pneumatic excitation may cause a membrane to deflect which may change a phase of the reflected ultrasound relative to a membrane that was not exposed to the pneumatic excitation. A deflection of the membrane may comprise a damped harmonic motion. This motion may be affected by changes in the elasticity of the membrane. A change in the membrane elasticity may occur, for example, if water, bacterial growth, or other foreign material is adjacent the membrane.

FIGS. 2A-2C shows the TM deflection and measured backing pressure in response to a series of positive and negative step function excitations applied to a healthy example TM. In FIG. 2A, a seal quality between the TM and the speculum may be of a sufficient quality. As shown, the deflection of the TM and the pressure behind the TM mirror the step function excitation that was applied. Some loss in pressure (and relaxation of the TM) is observed; however, the TM is generally responsive to the applied pressure.

In FIG. 2B, the seal between the TM and the speculum tip may only be a partial seal. As shown, the TM initially deflects in response to the pressure and the observed pressure changes; however, a significant loss of pressure and relaxation of the TM are observed. Further, an over correction in the neutral pressure portion of the sweep is observed.

In FIG. 2C, the seal between the TM and the speculum tip may be largely absent. As shown, the TM initially deflects in response to the pressure and the observed pressure changes; however, the response rapidly disappears. Further, the over correction in the neutral pressure portion of the sweep is almost as large as the initial response.

In an example pneumatic otoscopy measurement using reflected ultrasound, a pneumatic excitation may generate a movement of the surface or membrane during an interval of time. This interval may be coincident with acoustic wave delivered by an ultrasound transmitter to the surface or membrane. A pneumatic excitation may be continuous, may be pulsed, etc. The ultrasound reflected from the surface may be received at a transducer. A transducer may be the same transducer that generated the incident acoustic wave. A displacement of the surface or membrane may be related to a phase change in the received signal when compared to the transmit signal. A movement of the membrane may affect a phase change in the received ultrasound. A displacement may vary with time. An analysis of the temporal displacement of the surface or membrane, as measured by the phase shifts of the reflected ultrasound in response to the pneumatic excitation coupled to the surface or membrane may be used to determine the mechanical characteristics of the surface or membrane. The mechanical characteristics measured may include ductility, elasticity, hardness, etc. A non-contact measurement of the mechanical properties of a surface or alternatively a fluid below the surface of a membrane may be determined.

In some examples, an elasticity of a surface may be measured. The phase and/or amplitude of the reflected ultrasound from the membrane may be analyzed to produce an elasticity metric. The elasticity measurement may characterize a series of measurements in response to an applied excitation. The elasticity metric may be derived from the response of the surface and may provide an indication of one or more of several different phenomena. For example, the elasticity metric may indicate whether a surface adjacent to a membrane has a gaseous boundary or fluid boundary. For example, a membrane may move less, move more slowly, and or not move at all if the membrane has a fluid boundary. In an example, the elasticity metric may indicate, for the case of characterizing a fluid behind the membrane fluid boundary, the extent or a characteristic of the fluid. In some examples, the elasticity metric may be used to measure the characteristics of an elastic fluid with or without hysteresis of response. In a fluid with a hysteresis response, the fluid may exhibit an offset in displacement response, or “memory,” such that the response behavior in one direction is similar to the response behavior in the opposite direction, but only after traveling a particular displacement distance. For a hysteresis response, it may be necessary to characterize the linear behavior of the response after a particular measured displacement associated with the hysteresis of the system. A fluid elasticity metric may be determined from the characteristic response of the surface or membrane to the surface excitation and reflected ultrasound characterization.

In some examples, a surface deflection may be estimated. For example, the estimate of surface deflection may be derived from a measured estimate of velocity, acceleration, or any other metric associated with deflection over time. For example, a displacement of the surface will result in a shortened path from the transducer to the surface, and the reflected signal from the surface back to the transducer will return with a phase shift. The phase shift of the reflected ultrasound relative to an excitation thus confers information about an amount of deflection. With an estimate of the force applied by the excitation, an estimate of the elasticity of the membrane can be estimated.

Each excitation event may start a new deflection of the membrane. For example, an impulse excitation may pull the membrane in or push the membrane out for a limited period of time. For example, a square wave excitation may pull the membrane in or push the membrane out for a longer time. For example, a sine wave or other more complex excitation may be applied and the observed ringdown at the transducer may be a cross-correlation of the excitation field with the responding field. A pneumatic excitation may be applied at a frequency of less than 100 kHz, less than 1 kHz, less than 100 Hz, less than 10 Hz, less than 1 Hz, or less, or within a range given by any two the preceding values. A pneumatic excitation may be applied at a frequency greater than 1 Hz, greater than 10 Hz, greater than 100 Hz, greater than 1 kHz, greater than 100 kHz, or more, or within a range given by any two the preceding values. A pneumatic excitation may be applied within a range between 10 Hz and 100 Hz.

While FIGS. 2A-2C shows ultrasound pneumatic otoscopy data, improvements in a quality of a seal may also improve methods and systems for characterizing a membrane using optical coherence tomography (OCT). For example, a dynamic property of the tympanic membrane may comprise a phase delay or a time delay in the reflected optical signal in response to an applied pneumatic excitation. OCT may be used to collect depth dependent data related to the tympanic membrane. OCT may be used to collect frequency dependent data, such as wavelength of absorption of a membrane or a fluid adjacent a membrane.

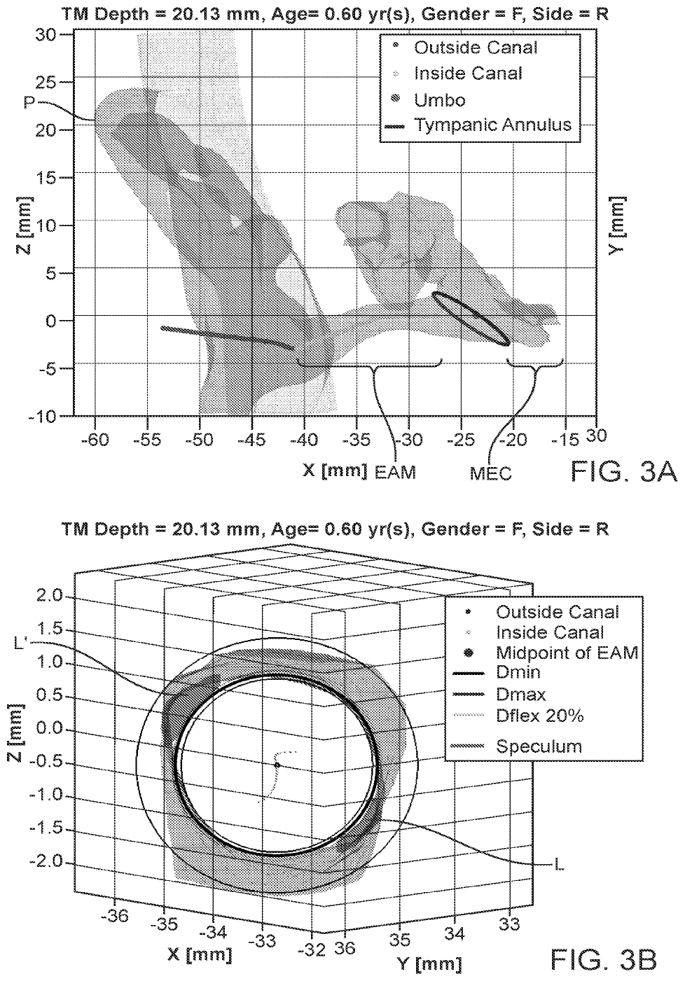

FIG. 3A depicts an anatomical model of an ear based on computerized tomography (CT) data of a right ear of a 0.6 year old female. The model shows the pinna P, the external auditory meatus EAM, and the middle ear cavity MEC. Also labeled is the tympanic annulus. As shown, the tympanic annulus is a 2D representation of the location of the TM in the shape of an ellipse. Within the tympanic annulus is a point indicating the location of the umbo, the portion of the TM where the malleus meets the membrane. A line indicating the geometric center of the EAM is also indicated. As shown, the EAM curves. The end of the center line of the EAM calculated by indentifying a point where the derivative of the radial distance sharply increases in magnitude, which indicates that ear has opened up to form the pinna. The second line segment indicates the center line of the pinna.

FIG. 3B depicts a cross section of ear model of FIG. 3A at a point within the EAM. As shown, the cross section of the EAM is irregular in shape. The mid-point of the EAM is indicated with four circles radiating outward from the indicated point. The innermost circle has a diameter Dmin, which is the circle defined by the diameter which contacts the walls of the EAM at their nearest points when the EAM is at its narrowest. The second circle from the inside is an outline of a distal-most circumference of a speculum. The third circle from the inside has a diameter of 20% larger than Dmin, as referred to herein as Dflex. Dflex may allow for deformability of the EAM, which may enlarge to adapt to insertion of a speculum. The percent increase in diameter of 20% is an empirical parameter. The fourth and outermost circle has a diameter Dmax, which is the circle defined by the diameter which contacts the walls of the EAM at their nearest points when the EAM is at its widest. The regions labeled L and L′ may be open when of the indicated dimeter is inserted and may yield a leak. The geometry of the speculum used, for example, a diameter, generally effects the area of the region(s) that yield a leak.

The devices, otoscopes, specula, and methods of use and manufacture thereof as disclosed herein address these leaks by providing a seal with one or more compressible members. The seal may improve a seal between an otoscope and a speculum. In some cases, the seal may create a static or constant fluid (e.g., air) between the external ear canal and the speculum tip, when the speculum is in position for observing the tympanic membrane. Providing a static or constant fluid may allow for control of a pressure within the ear canal. While other devices may be directed to keeping pressure changes from the outside from entering the ear canal (e.g., sound waves, water, etc.), the devices, otoscopes, specula, and methods of use and manufacture thereof as disclosed herein allow for a seal against both positive and negative pressure differentials. In particular, a seal may limit the air from escaping from the ear canal to the exterior of the speculum tip.

Various parameters may be addressed in order to produce a seal which produces an adequate barrier between the EAM and the exterior of the speculum. For example, the geometry of the cone of the speculum tip will affect a surface area which allows a leak. For example, the geometry of the one or more compressible members may be adapted to fit the geometry an ear. In some cases, a setback between a tip of the speculum and the one or more compressible members may improve the functionality the otoscope system by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A seal of the present disclosure may comprise a material that has a low coefficient of friction coefficient for insertion and removal during examination. A seal of the present disclosure may comprise a material that limits patient discomfort. A seal of the present disclosure may comprise a material that limits user (e.g. clinician) discomfort. A seal material may be selected in conjunction with seal geometry to increase a surface area of contact where force is applied. A seal of the present disclosure may comprise a material that is biocompatible. For example, portions of a seal which contact a patient or a user may be biocompatible. A biocompatible material may be characterized by and comply with the recommended endpoints of one or more of the following procedures: ISO 10993-1, 10993-2, 10993-3, 10993-4, 10993-5, 10993-6, 10993-7, 10993-8, 10993-9, 10993-10, and a material selected from USP Class VI.

A seal may comprise a soft polymeric material. A polymer material may comprise polydimethylsiloxane, polyester, a co-polyester, a polycarbonate, a thermoplastic polyurethane, a polypropylene, a polyethylene, a polypropylene, an acrylic, a cyclic block copolymer, a polyetherimide, a polyethersulfone, and a polytrimethylene terephthalate. A seal may comprise a styrenic block copolymer, a silicone rubber, an elastomeric alloy, a thermoplastic elastomer, a thermoplastic polymer, a natural rubber, a thermoplastic vulcanizate elastomer, a polyurethane elastomer, a block copolymer elastomer, a polyolefin blend elastomer, a thermoplastic co-polyester elastomer, a thermoplastic polyamide elastomer, or a combination thereof. A seal may comprise a synthetic rubber. A synthetic rubber may comprise butadiene rubber, butyl rubber, chlorosulfonated polyethylene, ethylene propylene diene monomer rubber, ethylene propylene rubber, fluroelastomer, nitrile rubber, perfluoroelastomer, polyacrylate rubber, neoprene rubber, polyisoprene, polysulfide rubber, polytetrafluroethylene, silicone rubber, styrene-butadiene rubber, and combinations thereof. A seal may comprise a thermoplastic polymer. A thermoplastic polymer may comprise a thermoplastic elastomer, a thermoplastic polyolefin, a thermoplastic polyurethane, a thermoplastic etheresterelastomers, a thermoplastic polyamide, a thermoplastic vulcanizate, etc. A thermoplastic polymer material may comprise polydimethyl siloxane.

A seal may comprise one or more compressible or deformable members. A compressibility or deformability may be characterized by hardness of the material. For example, a hardness may be characterized by a Shore durometer. A Shore durometer of a compressible or deformable member of the present disclosure may comprise a durometer measured on the Shore-alpha (e.g., Shore-A) scale. A Shore durometer of a compressible or deformable member of the present disclosure may be less than about 200, about 150, about 120, about 110, about 100, about 90, or less Shore-alpha. A Shore durometer of a compressible or deformable member of the present disclosure may be greater than about 10, about 20, about 30, about 40, about 50, about 60, about 70, or more Shore-alpha. A Shore durometer of a compressible or deformable member of the present disclosure may be within a range from about 50 to about 100, about 60 to about 150, about 20 to about 100, etc measured in Shore-alpha. A Shore durometer of a compressible or deformable member of the present disclosure may be within a range from about 60 to about 80 Shore-alpha, about 40 to about 80 Shore-alpha, about 30 to about 90 Shore-alpha, etc. A Shore durometer of a compressible or deformable member of the present disclosure may be about 70 Shore-alpha.

In some cases, a compressible or deformable member of the present disclosure is made of the same material as the seal. A compressible or deformable member of the present disclosure may comprise any material or combination of materials that a seal may comprise so long as a sufficient compressibility or deformability is established. In some cases, a compressible member is a separate part from the body of the seal. A seal which is releasably coupled may be easier to manufacture. A seal which is releasably coupled may be easier to fit to a subject ear.

In some examples, a seal may be releasably attached to a body of a speculum. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part. Releasable coupling may be aided by the addition of one or more retention features which secure the seal to the housing.

In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment. Movement off-axis may facilitate observation of speculum tip insertion to the external ear canal.

A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. A setback may be within a range from about 20 mm to about 1 mm, about 5 mm to about 15 mm, about 5 mm to about 7 mm, about 2 mm to about 5 mm, etc.

In some cases, a speculum may enter within an external auditory meatus at distance. For example, a speculum may enter within an external auditory meatus at distance greater than about 1 mm, about 2 mm, about 3 mm, about 4 mm, about 5 mm, about 6 mm, about 7 mm, about 8 mm, about 9 mm, or more. In some cases, a speculum may enter within an external auditory meatus at distance of less than about 20 mm, about 19 mm, about 18 mm, about 17 mm, about 16 mm, about 15 mm or less. In some cases, a speculum may enter within an external auditory meatus at distance of about 1 mm, about 2 mm, about 3 mm, about 4 mm, about 5 mm, about 6 mm, about 7 mm, about 8 mm, about 9 mm, etc. A speculum may enter within an external auditory meatus at distance within a range from about 20 mm to about 1 mm, about 5 mm to about 15 mm, about 5 mm to about 7 mm, about 2 mm to about 5 mm, etc.

In some cases, a compressible or deformable member of the present disclosure is configured to create a static or constant fluid (air) volume between the external ear canal and the speculum tip, when the speculum is in position for observing the tympanic membrane. For example, a compressible or deformable member may limit penetration of air between the ear canal and the exterior of the speculum. In some cases, the size and shape of the seal may be configured such that the compressible or deformable member substantially interfaces with the external auditory meatus to create a barrier to fluid or air flow. For example, the seal may provide a barrier between the lumen of the housing and an exterior of the housing not engaged with or within the external auditory meatus. In some cases, the seal is operably connected to a pneumatic otoscope comprising an indication that the pressure challenge wave within the lumen of the speculum is substantially retained within the lumen.

In some cases, the seal may retain a pressure within an external auditory meatus in response to a negative or positive pressure change. For example, the negative or positive pressure change may comprise a pressure challenge of a pneumatic otoscope, such as one provided as part of an otoscopy measurement, as disclosed herein. A seal may retain a pressure within an external auditory meatus in response to a negative or positive pressure change characterized by a magnitude of about 10 mm H₂O, about 20 mm H₂O, about 30 mm H₂O, about 40 mm H₂O, about 50 mm H₂O, about 60 mm H₂O, about 70 mm H₂O, about 80 mm H₂O, about 90 mm H₂O, about 100 mm H₂O, or an amount up to a pressure in which a patient TM would rupture. A seal may retain a pressure within an external auditory meatus in response to a negative or positive pressure change less than about a pressure in which a patient TM would rupture. A seal may retain a pressure within an external auditory meatus in response to a negative or positive pressure change greater than about 10 mm H₂O, about 20 mm H₂O, about 30 mm H₂O, about 40 mm H₂O, about 50 mm H₂O, about 60 mm H₂O, about 70 mm H₂O, or more. A seal may retain a pressure within an external auditory meatus in response to a negative or positive pressure change characterized by a magnitude within a range from about 10 mm H₂O to about 100 mm H₂O, about 20 mm H₂O to about 80 mm H₂O, about 50 mm H₂O to about 100 mm H₂O, about 30 mm H₂O to about 100 mm H₂O, etc.

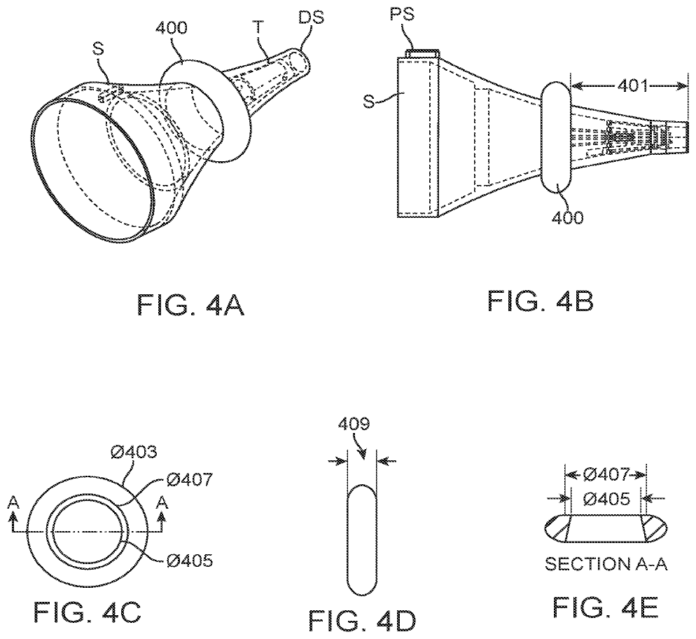

FIGS. 4A-4E illustrate an example of a seal with a single compressible member in the form of a torus. FIG. 4A illustrates an angled isometric view of an exterior of speculum comprising a seal 400. FIG. 4B shows a side view of a speculum S and seal 400. FIG. 4C shows a top view of a seal 400. FIG. 4D shows a section view of a seal 400. FIG. 4E shows a section view of a seal 400.

As shown in FIG. 4A and FIG. 4B, the speculum may comprise a pneumatic source PS which may be fluidically coupled to an interior volume of the speculum, e.g. a lumen. The pressure source PS may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein. FIG. 4A and FIG. 4B also illustrate a position of ultrasound transducer array T, which may be used to perform ultrasound pneumatic otoscopy measurements as described elsewhere herein.

The seal 400 may itself be a compressible member. Seal 400 comprises a single compressible member in the shape of a torus. Seal 400 may compress to form a barrier as disclosed herein. While a single compressible member is shown, a seal of the present disclosure may comprise a series of stacked torus shapes on the surface of the housing and along an axis of the transducer.

As shown in FIG. 4B, the seal may comprise a setback 401 from a most distal edge of the speculum DS. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

As shown in FIG. 4C, the seal may comprise a total width characterized by diameter 403. The diameter 403 of the seal 400 may be within an range from about 3 mm to about 40 mm, from about 3 mm to about 25 mm, from about 5 mm to about 20 mm, etc. The width of the seal may be greater than about 2 mm, about 3 mm, about 4 mm, about 5 mm, or more. The width of the seal may be less than about 40 mm, about 30 mm, about 20 mm, or less. The width of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 4C and FIG. 4E, the seal 400 may comprise a flat interior surface to aid in securing a seal to a housing of a speculum. In some cases, a seal 400 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

The seal 400 may comprise a proximal interior diameter 407 and a distal interior diameter 405. The distal interior diameter may smaller than diameter 403 by about 1 mm, 2 mm, about 3 mm, about 4 mm, about 5 mm, or more. The distal interior diameter may smaller than diameter 403 by less than about 40 mm, about 30 mm, about 20 mm, or less. The diameter 407 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 4D, the seal 400 may comprise a total length 409. The exterior profile of seal 400 may be spherical. In some cases, the exterior profile of seal 400 may be a distended sphere or an ellipse. A total length 409 may be greater than about 1 mm, about 2 mm, about 3 mm, about 4 mm, about 5 mm, about 6 mm, about 7 mm, about 8 mm, about 9 mm, or more. In some cases, a total length 409 may be less than about 20 mm, about 19 mm, about 18 mm, about 17 mm, about 16 mm, about 15 mm or less. In some cases, the total length is about 1 mm, about 2 mm, about 3 mm, about 4 mm, about 5 mm, about 6 mm, about 7 mm, about 8 mm, about 9 mm, etc. In the illustrated example, a total length 409 may be about 4 mm. A total length 409 may be within a range from about 20 mm to about 1 mm, about 5 mm to about 15 mm, about 5 mm to about 7 mm, about 2 mm to about 5 mm, etc.

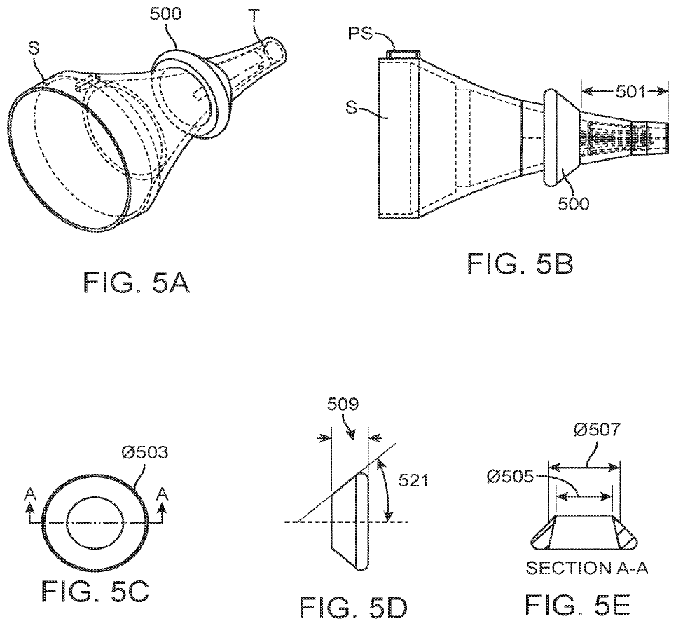

FIGS. 5A-4E illustrate an example of a seal with a single compressible member in the form of a cone. FIG. 5A illustrates an angled isometric view of an exterior of speculum comprising a seal 500. FIG. 5B shows a side view of a speculum S and seal 500. FIG. 5C shows a top view of a seal 500. FIG. 5D shows a section view of a seal 500. FIG. 5E shows a section view of a seal 500.

As shown in FIG. 5A and FIG. 5B, the speculum S may comprise a pneumatic source which may be fluidically coupled to an interior volume of the speculum, e.g. a lumen. The pressure source may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein. FIG. 5A and FIG. 5B also illustrate a position of ultrasound transducer array T, which may be used to perform ultrasound pneumatic otoscopy measurements as described elsewhere herein.

The seal 500 may itself be a compressible member. Seal 500 comprises a single compressible member in the shape of a cone. Seal 500 may compress to form a barrier as disclosed herein. While a single compressible member is shown, a seal of the present disclosure may comprise a series of stacked cone shapes on the surface of the housing and along an axis of the transducer.

As shown in FIG. 5B, the seal may comprise a setback 501 from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

As shown in FIG. 5C, the seal may comprise a total width characterized by diameter 503. The diameter 503 of the seal 500 may be within an range from about 3 mm to about 40 mm, from about 3 mm to about 25 mm, from about 5 mm to about 20 mm, etc. The width of the seal may be greater than about 2 mm, about 3 mm, about 4 mm, about 5 mm, or more. The width of the seal may be less than about 40 mm, about 30 mm, about 20 mm, or less. The width of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 5C and FIG. 5E, the seal 500 may comprise a flat interior surface to aid in securing a seal to a housing of a speculum. In some cases, a seal 500 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

The seal 500 may comprise a proximal interior diameter 507 and a distal interior diameter 505. The distal interior diameter 505 may be smaller than diameter 503 by about 1 mm, 2 mm, about 3 mm, about 4 mm, about 5 mm, or more. The distal interior diameter may smaller than diameter 503 by less than about 40 mm, about 30 mm, about 20 mm, or less. The distal interior diameter 505 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc. The proximal interior diameter 507 may smaller than diameter 503 by about 1 mm, 2 mm, about 3 mm, about 4 mm, about 5 mm, or more. The proximal interior diameter 507 may smaller than diameter 503 by less than about 40 mm, about 30 mm, about 20 mm, or less. The proximal interior diameter 507 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 5D, the seal 500 may comprise a total length 509. The exterior profile of seal 500 may be conical. A total length 509 may be greater than about 1 mm, about 2 mm, about 3 mm, about 4 mm, about 5 mm, about 6 mm, about 7 mm, about 8 mm, about 9 mm, or more. In some cases, a total length 509 may be less than about 20 mm, about 19 mm, about 18 mm, about 17 mm, about 16 mm, about 15 mm or less. In some cases, the total length is about 1 mm, about 2 mm, about 3 mm, about 4 mm, about 5 mm, about 6 mm, about 7 mm, about 8 mm, about 9 mm, etc. In the illustrated example, a total length 509 may be about 4 mm. A total length 509 may be within a range from about 20 mm to about 1 mm, about 5 mm to about 15 mm, about 5 mm to about 7 mm, about 2 mm to about 5 mm, etc.

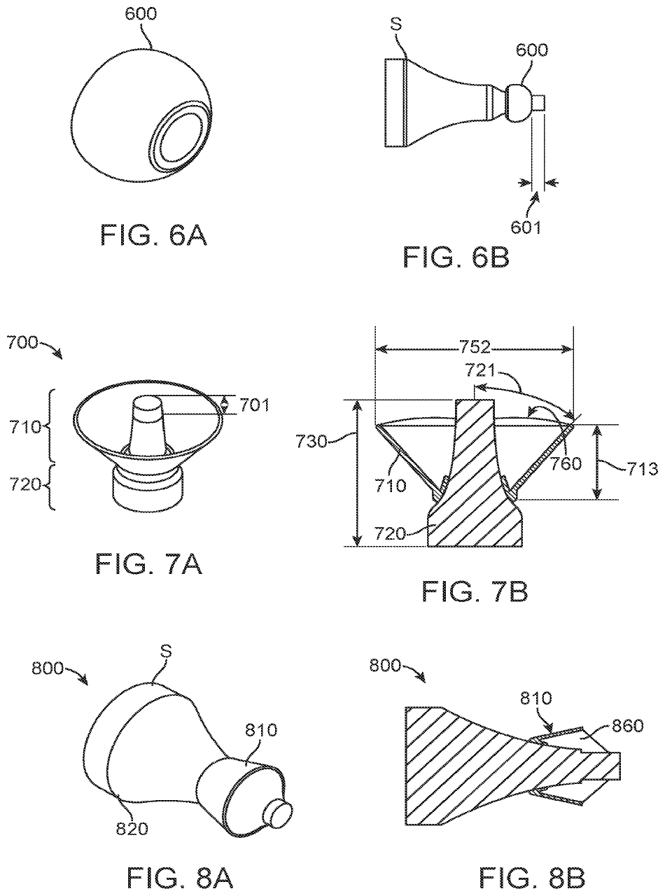

FIGS. 6A and 6B illustrate an example of a seal with a single compressible member 600 in the form of a sphere or a spherical cap. As shown in FIG. 6A and FIG. 6B, the speculum S may comprise a pneumatic source which may be fluidically coupled to an interior volume of the speculum, e.g. a lumen. The pressure source may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

The seal 600 may itself be a compressible member. Seal 600 comprises a single compressible member in the shape of a sphere. Seal 600 may compress to form a barrier as disclosed herein. While a single compressible member is shown, a seal of the present disclosure may comprise a series of stacked sphere shapes on the surface of the housing and along an axis of the transducer.

In some cases, compressible member 600 is coupled to a collar. The collar may comprise a cone configured to slide over a surface of a speculum. The collar may comprise one or more retention members on an interior lumen. In some cases, a seal 600 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

As shown in FIG. 6B, the seal may comprise a setback 601 from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

FIGS. 7A and 7B illustrate an example of a seal with a single compressible member in the form of an upturned cone. FIG. 7A is an exterior isometric view of a distal end of the seal 700. FIG. 7B shows a side section view of seal 700. Seal 700 may be coupled to an exterior of a speculum comprising a pneumatic source in a similar fashion to seal 1100 as disclosed herein.

The seal of 700 may comprise compressible member 710. The compressible member of seal 700 may comprise an upturned cone. In the example shown in FIG. 7A-7B, the seal may comprise 1 cone; however, other numbers of cones may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more compressible members.

Compressible member 710 may optionally comprise a gel 760 in an interior portion of the compressible member. A gel may be a stiff gel. The stiff gel may be fixed in place during manufacturing. In some cases, the gel is a fluid gel. The gel may be applied to a seal before insertion in an EAM. A gel may provide additional elastic restorative force to compressible member 710. Compressible member 710 may comprise a convex or concave profile, which may conform to an interior of an EAM. In some cases, the profile of compressible member 710 is not curved.

The seal 700 may also comprise collar 720. Collar 720 may comprise a cone configured to slide over a surface of a speculum. In some cases, a seal 700 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 720 may comprise a length. In some cases, the length of collar 720 may be greater than about 1 mm, about 2 mm, about 3 mm, about 4 mm, about 5 mm, about 6 mm, about 7 mm, about 8 mm, about 9 mm, or more. In some cases, the length may be less than about 20 mm, about 19 mm, about 18 mm, about 17 mm, about 16 mm, about 15 mm or less. In some cases, the length of collar 720 may be about 1 mm, about 2 mm, about 3 mm, about 4 mm, about 5 mm, about 6 mm, about 7 mm, about 8 mm, about 9 mm, etc. In some cases, the length may be within a range from about 20 mm to about 1 mm, about 5 mm to about 15 mm, about 5 mm to about 7 mm, about 2 mm to about 5 mm, etc.

As shown in FIG. 7A, the seal 700 may comprise a setback 701 from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback 701 may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. A setback may be within a range from about 20 mm to about 1 mm, about 5 mm to about 15 mm, about 5 mm to about 7 mm, about 2 mm to about 5 mm, etc.

As shown in FIG. 7B, the seal 700 may comprise a total length 730. A total length 730 may be greater than about 1 mm, about 2 mm, about 3 mm, about 4 mm, about 5 mm, about 6 mm, about 7 mm, about 8 mm, about 9 mm, or more. In some cases, a total length 730 may be less than about 20 mm, about 19 mm, about 18 mm, about 17 mm, about 16 mm, about 15 mm or less. In some cases, the total length 730 is about 1 mm, about 2 mm, about 3 mm, about 4 mm, about 5 mm, about 6 mm, about 7 mm, about 8 mm, about 9 mm, etc. A total length 730 may be within a range from about 20 mm to about 1 mm, about 5 mm to about 15 mm, about 5 mm to about 7 mm, about 2 mm to about 5 mm, etc.

FIG. 7B illustrates a length 713 of compressible member 710. The length of the compressible member 710 may be within an range from about 3 mm to about 40 mm, from about 3 mm to about 25 mm, from about 5 mm to about 20 mm, etc. The length of the compressible member 710 may be greater than about 2 mm, about 3 mm, about 4 mm, about 5 mm, or more. The length of the compressible member 710 may be less than about 40 mm, about 30 mm, about 20 mm, or less. The length of the compressible member 710 may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

The compressible member 710 may comprise an angled surface the distal surface. The compressible member may comprise an angled surface on the proximal surface. As shown, the angle of the distal and proximal surfaces may be about equal. Angle 721 of the distal surface may be less than about 90 degrees from the axis of the lumen. Angle 721 of the distal surface may be less than about 80 degrees, about 70 degrees, about 60 degrees, about 50 degrees, about 50 degrees, about 30 degrees, about 20 degrees or less from the axis of the lumen, wherein the angle is measured in a proximal direction on the axis. Angle 721 of the distal surface may be less than about 80 degrees, about 70 degrees, about 60 degrees, about 50 degrees, about 50 degrees, about 30 degrees, about 20 degrees or less from the axis of the lumen, wherein the angle is measured in a distal direction on the axis.

The compressible member 710 may have a width 752. The width 752 may be within an range from about 3 mm to about 40 mm, from about 3 mm to about 25 mm, from about 5 mm to about 20 mm, etc. The width 752 may be greater than about 2 mm, about 3 mm, about 4 mm, about 5 mm, or more. The width 752 may be less than about 40 mm, about 30 mm, about 20 mm, or less. The width 752 may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIGS. 8A and 8B illustrate an example of a seal with a single compressible member in the form of an upturned cone with a compressible gel. FIG. 8A is an exterior isometric view of a distal end of the seal 800. FIG. 8B is a side section view of seal 800. Seal 800 may be coupled to an exterior of a speculum comprising a pneumatic source in a similar fashion to seal 1100 as disclosed herein.

Seal 800 may comprise substantially similar dimensions to seal 700 but comprises a different gel arrangement. A gel 860 is filed to extend beyond the interior volume of the compressible member. A gel may be a stiff gel. The stiff gel may be fixed in place during manufacturing. A gel may provide additional elastic restorative force to compressible member 810.

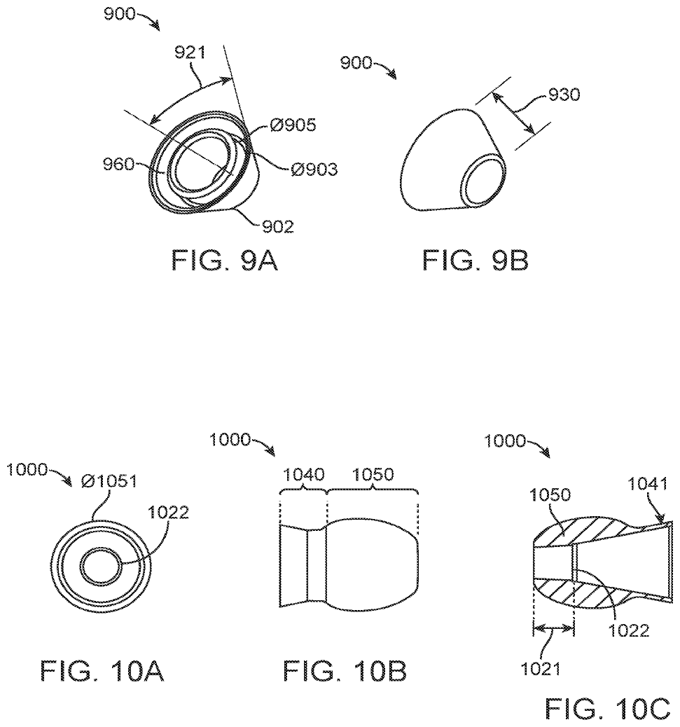

FIGS. 9A and 9B illustrate an example of a seal with a single compressible member in the form of a hollow cone. FIG. 9A is a rear isometric view of seal 900. FIG. 9B is a side isometric view of seal 900. Seal 900 may couple to a speculum, such as a speculum of an otoscope in a similar fashion to seal 500. The seal 900 comprise a compressible member 902. Seal 900 comprises a single compressible member in the shape of a cone. Seal 900 may compress to form a barrier as disclosed herein. While a single compressible member is shown, a seal of the present disclosure may comprise a series of stacked cone shapes on the surface of the housing and along an axis of the transducer.

The seal may comprise a setback (not shown) from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

As shown in FIG. 9A, the seal may comprise a total width characterized by diameter 903. The diameter 903 of the seal 500 may be within an range from about 3 mm to about 40 mm, from about 3 mm to about 25 mm, from about 5 mm to about 20 mm, etc. The width of the seal may be greater than about 2 mm, about 3 mm, about 4 mm, about 5 mm, or more. The width of the seal may be less than about 40 mm, about 30 mm, about 20 mm, or less. The width of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

The seal 900 may comprise a flat interior surface to aid in securing a seal to a housing of a speculum. In some cases, a seal 900 may be releasably coupled to a speculum. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

The seal 900 may comprise interior diameter 905. The interior diameter 905 may be smaller than diameter 903 by about 1 mm, 2 mm, about 3 mm, about 4 mm, about 5 mm, or more. The interior diameter may smaller than diameter 903 by less than about 40 mm, about 30 mm, about 20 mm, or less. The interior diameter 905 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

Compressible member 902 may optionally comprise a gel 960 in an interior portion of the compressible member. A gel may be a stiff gel. The stiff gel may be fixed in place during manufacturing. In some cases, the gel is a fluid gel. The gel may be applied to a seal before insertion in an EAM. A gel may provide additional elastic restorative force to compressible member 902. Compressible member 902 may comprise a convex or concave profile, which may conform to an interior of an EAM. In some cases, the profile of compressible member 902 is not curved.

As shown in FIG. 9B, the seal 900 may comprise a total length 930. The exterior profile of seal 900 may be conical. A total length 930 may be greater than about 1 mm, about 2 mm, about 3 mm, about 4 mm, about 5 mm, about 6 mm, about 7 mm, about 8 mm, about 9 mm, or more. In some cases, a total length 930 may be less than about 20 mm, about 19 mm, about 18 mm, about 17 mm, about 16 mm, about 15 mm or less. In some cases, the total length 930 is about 1 mm, about 2 mm, about 3 mm, about 4 mm, about 5 mm, about 6 mm, about 7 mm, about 8 mm, about 9 mm, etc. In the illustrated example, a total length 509 may be about 4 mm. A total length 930 may be within a range from about 20 mm to about 1 mm, about 5 mm to about 15 mm, about 5 mm to about 7 mm, about 2 mm to about 5 mm, etc.

FIGS. 10A-10C illustrate an example of a seal with a single compressible member in the form of a bulb. FIG. 10A is a rear view showing a proximal end of seal 1000. FIG. 10B is a side exterior view of a seal 1000. FIG. 10C is a side section view of seal 1000.

As shown in FIG. 10B, the seal 1000 may comprise a compressible member 1050. The compressible member of seal 1000 may comprise a bulb shape. In the example shown in FIG. 10A and FIG. 10B, the seal may comprise 1 bulb; however, other numbers of bulbs may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more compressible members.

The seal 1000 may also comprise collar 1040. Collar 1040 may comprise a cone configured to slide over a surface of a speculum. In some cases, a seal 1000 may be releasably coupled to a speculum. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1040 may comprise a length. In some cases, the length of collar 1040 may be greater than about 1 mm, about 2 mm, about 3 mm, about 4 mm, about 5 mm, about 6 mm, about 7 mm, about 8 mm, about 9 mm, or more. In some cases, the length may be less than about 20 mm, about 19 mm, about 18 mm, about 17 mm, about 16 mm, about 15 mm or less. In some cases, the length 1140 may be about 1 mm, about 2 mm, about 3 mm, about 4 mm, about 5 mm, about 6 mm, about 7 mm, about 8 mm, about 9 mm, etc. In some cases, the length may be within a range from about 20 mm to about 1 mm, about 5 mm to about 15 mm, about 5 mm to about 7 mm, about 2 mm to about 5 mm, etc.

Compressible member 1050 may comprise a length. In some cases, the length of Compressible member 1050 may be greater than about 1 mm, about 2 mm, about 3 mm, about 4 mm, about 5 mm, about 6 mm, about 7 mm, about 8 mm, about 9 mm, or more. In some cases, the length may be less than about 20 mm, about 19 mm, about 18 mm, about 17 mm, about 16 mm, about 15 mm or less. In some cases, the length 1050 may be about 1 mm, about 2 mm, about 3 mm, about 4 mm, about 5 mm, about 6 mm, about 7 mm, about 8 mm, about 9 mm, etc. In some cases, the length may be within a range from about 20 mm to about 1 mm, about 5 mm to about 15 mm, about 5 mm to about 7 mm, about 2 mm to about 5 mm, etc.

The seal 1000 may comprise a setback (not shown) from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback may be characterized as a distance from a most distal of the one or more compressible members to a most distal edge of the speculum. In some cases, the compressible member is flush with an end of the speculum tip. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. A setback may be within a range from about 20 mm to about 1 mm, about 5 mm to about 15 mm, about 5 mm to about 7 mm, about 2 mm to about 5 mm, etc.

As shown in FIG. 10A, the compressible member 1050 may have a width 1051. The width 1051 may be within an range from about 3 mm to about 40 mm, from about 3 mm to about 25 mm, from about 5 mm to about 20 mm, etc. The width 1051 may be greater than about 2 mm, about 3 mm, about 4 mm, about 5 mm, or more. The width 1051 may be less than about 40 mm, about 30 mm, about 20 mm, or less. The width 1051 may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 10C illustrates a section view with retention members within an interior of the seal 1000. In some cases, a seal 1000 may be releasably coupled to a speculum. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

As shown in FIG. 10C, a seal may comprise a plurality of retention members. The collar may comprise one or more retention members on an interior lumen. In the example shown in FIG. 10C, the seal may comprise 3 retention members; however, other numbers of retention members may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more retention members.

The most distal and the central retention 1022 member may be separated by a distance 1021. The distance 1021 may be within a range from about 20% to about 80% of the total length of the seal 1000. The distance 1021 may be about 10%, about 20%, about 30%, about 40%, about 50%, about 60%, about 70%, about 80%, about 90%, etc. of the total length of the seal 1000.

As shown in FIG. 10C, a seal may comprise a variable wall thickness. In the illustrated embodiment thicknesses at locations 1041 is indicated. The wall thickness at location 1041 may be about 0.5 mm. The thickness of the seal 1000 may be variable to create the compressible member 1050. In some cases, the thickness of the seal 1000 may be narrow at a proximal end to allow the seal to couple to a speculum.

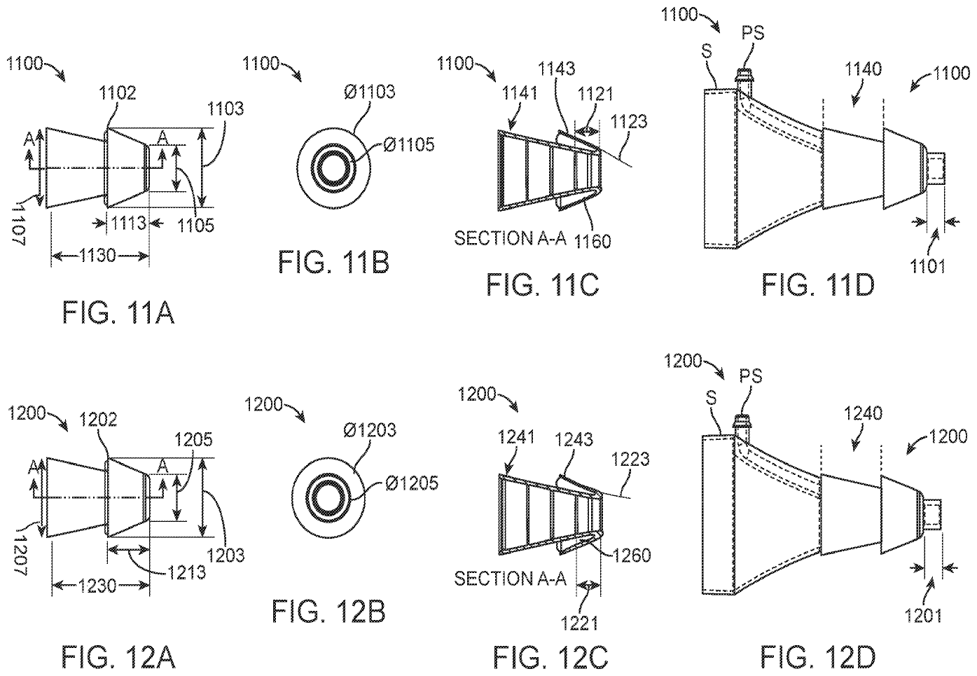

FIGS. 11A-11C illustrate an example of a seal with a collar and a single compressible member in the form of a convex cone. FIG. 11A illustrates a side exterior view of a seal 1100. FIG. 11B illustrates a front view of a seal 1100. FIG. 11C illustrates a section view of a seal 1100 along an axis A-A. FIG. 11D illustrates a side isometric view of an exterior of speculum comprising a seal 1100.

As shown in FIG. 11D, the speculum S may comprise a pneumatic source PS which may be fluidically coupled to an interior volume of the speculum, e.g. a lumen. The pressure source PS may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

The seal of 1100 may comprise compressible member 1102. The compressible member of seal 1100 may comprise a flange. In the example shown in FIG. 11A-11C, the seal may comprise 1 flange; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more compressible members.

Compressible member 1102 may optionally comprise a gel in an interior portion of the compressible member. A gel may be a stiff gel. The stiff gel may be fixed in place during manufacturing. In some cases, the gel is a fluid gel. The gel may be applied to a seal before insertion in an EAM. A gel may provide additional elastic restorative force to compressible member 1102. Compressible member 1102 may comprise a convex profile 1123, which may conform to an interior of an EAM.

The seal 1100 may also comprise collar 1140. Collar 1140 may comprise a cone configured to slide over a surface of a speculum. In some cases, a seal 1100 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1140 may comprise a length. In some cases, the length 1140 may be greater than about 1 mm, about 2 mm, about 3 mm, about 4 mm, about 5 mm, about 6 mm, about 7 mm, about 8 mm, about 9 mm, or more. In some cases, the length 1140 may be less than about 20 mm, about 19 mm, about 18 mm, about 17 mm, about 16 mm, about 15 mm or less. In some cases, the length 1140 may be about 1 mm, about 2 mm, about 3 mm, about 4 mm, about 5 mm, about 6 mm, about 7 mm, about 8 mm, about 9 mm, etc. In some cases, the length 1140 may be within a range from about 20 mm to about 1 mm, about 5 mm to about 15 mm, about 5 mm to about 7 mm, about 2 mm to about 5 mm, etc.

As shown in FIG. 11D, the seal 1100 may comprise a setback 1101 from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback 1101 may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. A setback may be within a range from about 20 mm to about 1 mm, about 5 mm to about 15 mm, about 5 mm to about 7 mm, about 2 mm to about 5 mm, etc.

As shown in FIG. 11A, the seal 1100 may comprise a total length 1130. A total length 1130 may be greater than about 1 mm, about 2 mm, about 3 mm, about 4 mm, about 5 mm, about 6 mm, about 7 mm, about 8 mm, about 9 mm, or more. In some cases, a total length 1130 may be less than about 20 mm, about 19 mm, about 18 mm, about 17 mm, about 16 mm, about 15 mm or less. In some cases, the total length 1130 is about 1 mm, about 2 mm, about 3 mm, about 4 mm, about 5 mm, about 6 mm, about 7 mm, about 8 mm, about 9 mm, etc. A total length 1130 may be within a range from about 20 mm to about 1 mm, about 5 mm to about 15 mm, about 5 mm to about 7 mm, about 2 mm to about 5 mm, etc.

FIG. 11A illustrates a length 1113 of compressible member 1102. The length of the compressible member 1102 may be within an range from about 3 mm to about 40 mm, from about 3 mm to about 25 mm, from about 5 mm to about 20 mm, etc. The length of the compressible member 1102 may be greater than about 2 mm, about 3 mm, about 4 mm, about 5 mm, or more. The length of the compressible member 1102 may be less than about 40 mm, about 30 mm, about 20 mm, or less. The length of the compressible member 1102 may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 11A illustrates an axis of symmetry A-A. In some cases, the seal may be radially symmetric about an axis. In some cases, a seal may not be radially symmetric.

FIG. 11B illustrates a front exterior view of the distal end of seal 1100. In the illustrated example, the width of the seal 1107 may be equal to the diameter 1103 of the compressible member 1102. The width 1107 of the seal may be within an range from about 3 mm to about 40 mm, from about 3 mm to about 25 mm, from about 5 mm to about 20 mm, etc. The width 1107 of the seal may be greater than about 2 mm, about 3 mm, about 4 mm, about 5 mm, or more. The width of the seal may be less than about 40 mm, about 30 mm, about 20 mm, or less. The width 1107 of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 11B, the compressible members 1102 may have may comprise a proximal exterior diameter 1103 and a distal exterior diameter 1105. The distal exterior diameter 1105 may smaller than width 1107 by about 1 mm, 2 mm, about 3 mm, about 4 mm, about 5 mm, or more. The distal exterior diameter may smaller than width 1107 by less than about 40 mm, about 30 mm, about 20 mm, or less. The distal exterior diameter 1105 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc. The proximal exterior diameter 1103 may smaller than width 1107 by about 1 mm, 2 mm, about 3 mm, about 4 mm, about 5 mm, or more. The proximal exterior diameter 1103 may smaller than width 1107 by less than about 40 mm, about 30 mm, about 20 mm, or less. The proximal exterior diameter 1103 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 11C illustrates a section view with retention members within an interior of the seal 1100. In some cases, a seal 1100 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

As shown in FIG. 11C, a seal may comprise a plurality of retention members. The collar may comprise one or more retention members on an interior lumen. In the example shown in FIG. 11C, the seal may comprise 5 retention members; however, other numbers of retention members may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more retention members.

The most distal and the central retention member may be separated by a distance 1121. The distance 1121 may be within a range from about 20% to about 80% of the distance 1130. The distance 1121 may be about 10%, about 20%, about 30%, about 40%, about 50%, about 60%, about 70%, about 80%, about 90%, etc. of the distance 1130.

As shown in FIG. 11C, a seal may comprise an increasing wall thickness. In the illustrated embodiment thicknesses at two locations 1141 and 1143 are indicated. The wall thickness at location 1141 may be about 0.5 mm. The flange thickness at location 1143 may be about 0.25 mm. An increasing wall thickness may provide structural support while aiding in retention of the seal on the speculum.

FIGS. 12A-12C illustrate an example of a seal with a collar and a single compressible member in the form of a concave cone. FIG. 12A illustrates a side exterior view of a seal 1200. FIG. 12B illustrates a front view of a seal 1200. FIG. 12C illustrates a section view of a seal 1200 along an axis A-A. FIG. 12D illustrates a side isometric view of an exterior of speculum comprising a seal 1200.

As shown in FIG. 12D, the speculum S may comprise a pneumatic source PS which may be fluidically coupled to an interior volume of the speculum, e.g. a lumen. The pressure source PS may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

The seal of 1200 may comprise compressible member 1202. The compressible member of seal 1200 may comprise a flange. In the example shown in FIG. 12A-12C, the seal may comprise 1 flange; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more flanges.

Compressible member 1202 may optionally comprise a gel in an interior portion of the compressible member. A gel may be a stiff gel. The stiff gel may be fixed in place during manufacturing. In some cases, the gel is a fluid gel. The gel may be applied to a seal before insertion in an EAM. A gel may provide additional elastic restorative force to compressible member 1202. Compressible member 1202 may comprise a concave profile 1223, which may conform to an interior of an EAM.

The seal 1200 may also comprise collar 1240. Collar 1240 may comprise a cone configured to slide over a surface of a speculum. In some cases, a seal 1200 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1240 may comprise a length. In some cases, the length 1240 may be greater than about 1 mm, about 2 mm, about 3 mm, about 4 mm, about 5 mm, about 6 mm, about 7 mm, about 8 mm, about 9 mm, or more. In some cases, the length 1240 may be less than about 20 mm, about 19 mm, about 18 mm, about 17 mm, about 16 mm, about 15 mm or less. In some cases, the length 1240 may be about 1 mm, about 2 mm, about 3 mm, about 4 mm, about 5 mm, about 6 mm, about 7 mm, about 8 mm, about 9 mm, etc. In some cases, the length 1240 may be within a range from about 20 mm to about 1 mm, about 5 mm to about 15 mm, about 5 mm to about 7 mm, about 2 mm to about 5 mm, etc.

As shown in FIG. 12D, the seal 1200 may comprise a setback 1201 from a most distal edge of the speculum. In some cases, a setback between a tip of the speculum and the one or more compressible members may improve the functionality the otoscope system, as described herein, for example, by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

A setback 1201 may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. A setback may be within a range from about 20 mm to about 1 mm, about 5 mm to about 15 mm, about 5 mm to about 7 mm, about 2 mm to about 5 mm, etc.

As shown in FIG. 12A, the seal 1200 may comprise a total length 1230. A total length 1230 may be greater than about 1 mm, about 2 mm, about 3 mm, about 4 mm, about 5 mm, about 6 mm, about 7 mm, about 8 mm, about 9 mm, or more. In some cases, a total length 1230 may be less than about 20 mm, about 19 mm, about 18 mm, about 17 mm, about 16 mm, about 15 mm or less. In some cases, the total length 1230 is about 1 mm, about 2 mm, about 3 mm, about 4 mm, about 5 mm, about 6 mm, about 7 mm, about 8 mm, about 9 mm, etc. A total length 1230 may be within a range from about 20 mm to about 1 mm, about 5 mm to about 15 mm, about 5 mm to about 7 mm, about 2 mm to about 5 mm, etc.

FIG. 12A illustrates a length 1213 of compressible member 1202. The length of the compressible member 1202 may be within an range from about 3 mm to about 40 mm, from about 3 mm to about 25 mm, from about 5 mm to about 20 mm, etc. The length of the compressible member 1202 may be greater than about 2 mm, about 3 mm, about 4 mm, about 5 mm, or more. The length of the compressible member 1202 may be less than about 40 mm, about 30 mm, about 20 mm, or less. The length of the compressible member 1202 may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 12A illustrates an axis of symmetry A-A. In some cases, the seal may be radially symmetric about an axis. In some cases, a seal may not be radially symmetric.

FIG. 12B illustrates a front exterior view of the distal end of seal 1200. In the illustrated example, the width of the seal 1207 may be equal to the diameter 1203 of the compressible member 1202. The width 1207 of the seal may be within an range from about 3 mm to about 40 mm, from about 3 mm to about 25 mm, from about 5 mm to about 20 mm, etc. The width 1207 of the seal may be greater than about 2 mm, about 3 mm, about 4 mm, about 5 mm, or more. The width of the seal may be less than about 40 mm, about 30 mm, about 20 mm, or less. The width 1207 of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 12B, the compressible members 1202 may have may comprise a proximal exterior diameter 1203 and a distal exterior diameter 1205. The distal exterior diameter 1205 may smaller than width 1207 by about 1 mm, 2 mm, about 3 mm, about 4 mm, about 5 mm, or more. The distal exterior diameter may smaller than width 1207 by less than about 40 mm, about 30 mm, about 20 mm, or less. The distal exterior diameter 1205 of the seal may be about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc. The proximal exterior diameter 1203 may smaller than width 1207 by about 1 mm, 2 mm, about 3 mm, about 4 mm, about 5 mm, or more. The proximal exterior diameter 1203 may smaller than width 1207 by less than about 40 mm, about 30 mm, about 20 mm, or less. The proximal exterior diameter 1203 of the seal may be about 2 mm, about 3 mm, about 4 mm, about 5 mm, about 6 mm, about 7 mm, about 8 mm, about 9 mm, about 10 mm, about 12 mm, about 12 mm, about 13 mm, about 14 mm, about 15 mm, about 16 mm, about 17 mm, about 18 mm, about 19 mm, etc.

FIG. 12C illustrates a section view with retention members within an interior of the seal 1200. In some cases, a seal 1200 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

As shown in FIG. 12C, a seal may comprise a plurality of retention members. The collar may comprise one or more retention members on an interior lumen. In the example shown in FIG. 12C, the seal may comprise 5 retention members; however, other numbers of retention members may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more retention members.

The most distal and the central retention member may be separated by a distance 1221. The distance 1221 may be within a range from about 20% to about 80% of the distance 1230. The distance 1221 may be about 10%, about 20%, about 30%, about 40%, about 50%, about 60%, about 70%, about 80%, about 90%, etc. of the distance 1230.

As shown in FIG. 12C, a seal may comprise an increasing wall thickness. In the illustrated embodiment thicknesses at two locations 1241 and 1243 are indicated. The wall thickness at location 1241 may be about 0.5 mm. The flange thickness at location 1243 may be about 0.25 mm. An increasing wall thickness may provide structural support while aiding in retention of the seal on the speculum.

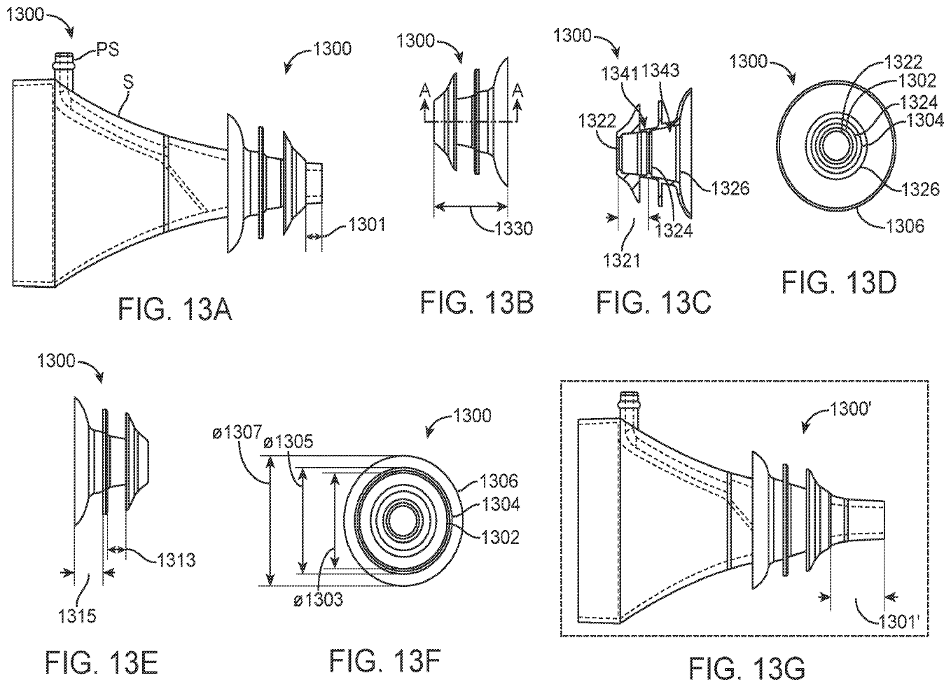

FIGS. 13A-13G illustrate an example of a seal with three flanges of varying diameter. FIG. 13A illustrates a side view of a speculum S and a seal 1300. FIG. 13B illustrates a side exterior view of seal 1300. FIG. 13C illustrates a side section view of seal 1300. FIG. 13D illustrates a rear view of the proximal end of seal 1300. FIG. 13E illustrates a side exterior view of seal 1300 on an opposite side from FIG. 13B. FIG. 13F illustrates a front exterior view of the distal end of seal 1300.

As shown in FIG. 13A, the speculum may comprise a pneumatic source PS which may be fluidically coupled to an interior volume of the speculum, e.g. a lumen. The pressure source PS may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

The seal of 1300 may comprise one or more compressible members 1302, 1304, and 1306. The one or more compressible members of seal 1300 may comprise flanges. In the example shown in FIG. 13A-G, the seal may comprise 3 flanges; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more flanges.

Each of the flanges of seal 1300 comprises a different geometry. Compressible member 1302 may be the most distal compressible member. A most distal compressible member 1302 may comprise a flat taper on the distal surface. A most distal compressible member 1302 may comprise a flat surface on the proximal surface. Compressible member 1306 may the most proximal compressible member. A most proximal compressible member 1306 may comprise a curved taper on the distal surface. A most proximal compressible member 1306 may comprise a curved tapered surface on the proximal surface. The middle compressible member 1304 may comprise flax proximal and distal surfaces.

As shown in FIG. 13A, the seal may comprise a setback 1301 from a most distal edge of the speculum DS. A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

FIG. 13G illustrates an example of seal 1300′ comprising a longer setback 1301′. In the illustrated example, the setback may be about 7 mm. In some cases, a setback between a tip of the speculum and the one or more compressible members may improve the functionality the otoscope system by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

As shown in FIG. 13B, the seal may comprise a total length 1330. The length of the seal may be within an range from about 3 mm to about 40 mm, from about 3 mm to about 25 mm, from about 5 mm to about 20 mm, etc. The length of the seal may be greater than about 2 mm, about 3 mm, about 4 mm, about 5 mm, or more. The length of the seal may be less than about 40 mm, about 30 mm, about 20 mm, or less. The length of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 13B illustrates an axis of symmetry A-A. In some cases, the seal may be radially symmetric about an axis. In some cases, a seal may not be radially symmetric.

FIG. 13C illustrates a section view with retention members within an interior of the seal 1300. In some cases, a seal 1300 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

As shown in FIG. 13C, a seal may comprise a plurality of retention members, 1322, 1324, and 1326. In the example shown in FIG. 13C, the seal may comprise 3 retention members; however, other numbers of retention members may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more retention members. The most distal and the central retention member may be separated by a distance 1321. The distance 1321 may be within a range from about 20% to about 80% of the distance 1330. The distance 1321 may be about 10%, about 20%, about 30%, about 40%, about 50%, about 60%, about 70%, about 80%, about 90%, etc. of the distance 1330.

As shown in FIG. 13C, a seal may comprise an increasing wall thickness. In the illustrated embodiment a wall thickness at two locations 1341 and 1343 are indicated. The wall thickness at location 1341 may be about 0.25 mm. The wall thickness at location 1341 may be about 0.5 mm. An increasing wall thickness may provide structural support while aiding in retention of the seal on the speculum.

FIG. 13D illustrates a rear view of the proximal end of seal 1300. As shown in FIG. 13D, the plurality of retention members, 1322, 1324, and 1326 may comprise a radially increasing diameter from 1322 to 1326. The plurality of compressible members 1302, 1304, 1306 may have a radially increasing interior diameter from 1302 to 1306.

As shown in FIG. 13E, the one or more compressible members may be spaced by distances 1313 and 1315. Distance 1313 may be a distance between compressible members 1302 and 1304. Distance 1315 may be a distance between compressible members 1304 and 1306. The distance 1313 may be within a range from about 20% to about 80% of the distance 1330. The distance 1313 may be about 10%, about 20%, about 30%, about 40%, about 50%, about 60%, about 70%, about 80%, about 90%, etc. of the distance 1330. The distance 1315 may be within a range from about 20% to about 80% of the distance 1330. The distance 1315 may be about 10%, about 20%, about 30%, about 40%, about 50%, about 60%, about 70%, about 80%, about 90%, etc. of the distance 1330.

FIG. 13F illustrates a front exterior view of the distal end of seal 1300. In the illustrated example, the width of the seal may be equal to the diameter 1307 of the most proximal compressible member 1306. The width of the seal may be within an range from about 3 mm to about 40 mm, from about 3 mm to about 25 mm, from about 5 mm to about 20 mm, etc. The width of the seal may be greater than about 2 mm, about 3 mm, about 4 mm, about 5 mm, or more. The width of the seal may be less than about 40 mm, about 30 mm, about 20 mm, or less. The width of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 13F, the plurality of compressible members 1302, 1304, 1306 may have a radially increasing exterior diameters from 1302 to 1306. While in some cases the diameters of the compressible members may not me monotonically increasing from 1302 to 1306, the diameters of the compressible members in the illustrated examples are monotonically increasing from 1302 to 1306.

The compressible members may have diameters 1303, 1305, and 1307. The diameter 1303 may be the diameter of the most distal compressible member 1302. The diameter 1307 may be the diameter of the most proximal compressible member 1306. The diameter 1305 may be the diameter of the middle compressible member 1304. The diameter 1303 may be within a range from about 20% to about 80% of the diameter 1307. The diameter 1303 may be about 10%, about 20%, about 30%, about 40%, about 50%, about 60%, about 70%, about 80%, about 90%, etc. of the diameter 1307. The diameter 1305 may be within a range from about 20% to about 80% of the diameter 1307. The diameter 1305 may be about 10%, about 20%, about 30%, about 40%, about 50%, about 60%, about 70%, about 80%, about 90%, etc. of the diameter 1307.

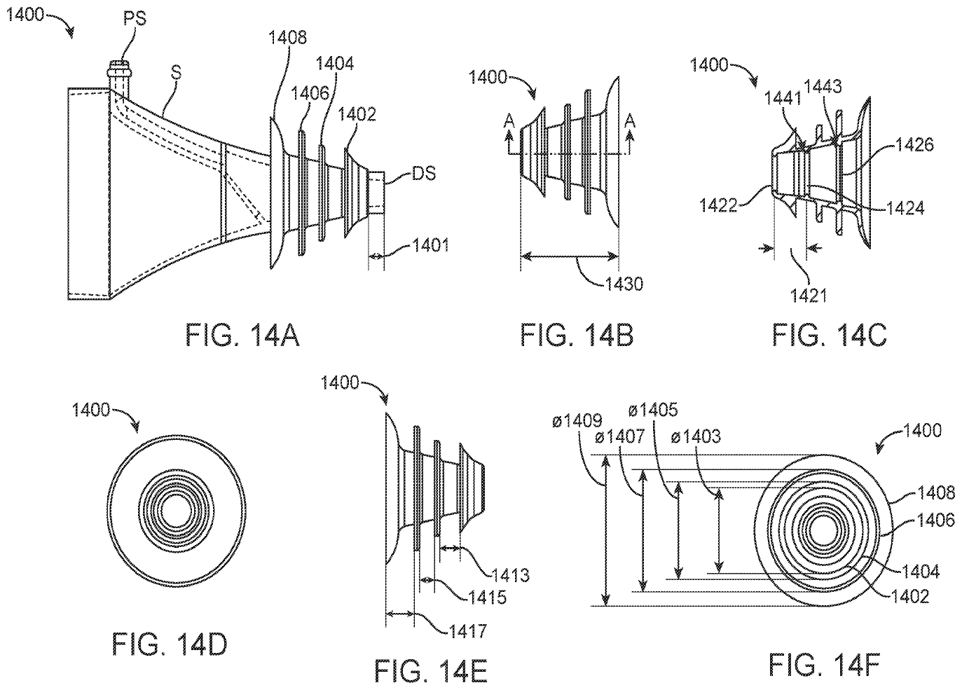

FIGS. 14A-14F illustrate an example of a seal with four flanges of varying diameter. FIG. 14A illustrates a side view of a speculum S and a seal 1400. FIG. 14B illustrates a side exterior view of seal 1400. FIG. 14C illustrates a side section view of seal 1400. FIG. 14D illustrates a rear view of the proximal end of seal 1400. FIG. 14E illustrates a side exterior view of seal 1400 on an opposite side from FIG. 14B. FIG. 14F illustrates a front exterior view of the distal end of seal 1400.

As shown in FIG. 14A, the speculum may comprise a pneumatic source PS which may be fluidically coupled to an interior volume of the speculum, e.g. a lumen. The pressure source PS may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

The seal of 1400 may comprise one or more compressible members 1402, 1404, 1406, and 1408. The one or more compressible members of seal 1400 may comprise flanges. In the example shown in FIG. 14A-F, the seal may comprise 4 flanges; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more flanges.

Each of the flanges of seal 1400 comprises a different geometry. Compressible member 1402 may be the most distal compressible member. A most distal compressible member 1402 may comprise a flat taper on the distal surface. A most distal compressible member 1402 may comprise a flat surface on the proximal surface. Compressible member 1408 may the most proximal compressible member. A most proximal compressible member 1408 may comprise a curved taper on the distal surface. A most proximal compressible member 1408 may comprise a curved tapered surface on the proximal surface. The middle compressible members 1404 and 1406 may comprise flax proximal and distal surfaces.

As shown in FIG. 14A, the seal may comprise a setback 1401 from a most distal edge of the speculum DS. A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

In some cases, a setback between a tip of the speculum and the one or more compressible members may improve the functionality the otoscope system by allowing for penetration of the speculum tip into the external ear canal and while allowing the operator to manipulate the speculum tip off the center axis of the ear canal. For example, the setback may allow for manipulation of the speculum tip in a radial direction. For example, the setback may allow for pitch, yaw, and roll of the speculum tip. Movement off-axis may facilitate the process of visualizing the cone of light, such as allowing for movement of cerumen or allowing for optical alignment.

As shown in FIG. 14B, the seal may comprise a total length 1430. The length of the seal may be within an range from about 3 mm to about 40 mm, from about 3 mm to about 25 mm, from about 5 mm to about 20 mm, etc. The length of the seal may be greater than about 2 mm, about 3 mm, about 4 mm, about 5 mm, or more. The length of the seal may be less than about 40 mm, about 30 mm, about 20 mm, or less. The length of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 14B illustrates an axis of symmetry A-A. In some cases, the seal may be radially symmetric about an axis. In some cases, a seal may not be radially symmetric.

FIG. 14C illustrates a section view with retention members within an interior of the seal 1400. In some cases, a seal 1400 may be releasably coupled to speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

As shown in FIG. 14C, a seal may comprise a plurality of retention members, 1422, 1424, 1426, and 1428. In the example shown in FIG. 14C, the seal may comprise 4 retention members; however, other numbers of retention members may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more retention members. The most distal and the next most distal retention member may be separated by a distance 1421. The distance 1421 may be within a range from about 20% to about 80% of the distance 1430. The distance 1421 may be about 10%, about 20%, about 30%, about 40%, about 50%, about 60%, about 70%, about 80%, about 90%, etc. of the distance 1430.

As shown in FIG. 14C, a seal may comprise an increasing wall thickness. In the illustrated embodiment a wall thickness at two locations 1441 and 1443 are indicated. The wall thickness at location 1441 may be about 0.25 mm. The wall thickness at location 1441 may be about 0.5 mm. An increasing wall thickness may provide structural support while aiding in retention of the seal on the speculum.

FIG. 14D illustrates a rear view of the proximal end of seal 1400. As shown in FIG. 14D, the plurality of retention members, 1422, 1424, 1426, and 1428 may comprise a radially increasing diameter from 1422 to 1428. The plurality of compressible members 1402, 1404, 1406, and 1408 may have a radially increasing interior diameter from 1402 to 1408.

As shown in FIG. 14E, the one or more compressible members may be spaced by distances 1413, 1415, and 1417. Distance 1413 may be a distance between compressible members 1402 and 1404. Distance 1415 may be a distance between compressible members 1404 and 1406. Distance 1417 may be a distance between compressible members 1406 and 1408. The distance 1413 may be within a range from about 20% to about 80% of the distance 1430. The distance 1413 may be about 10%, about 20%, about 30%, about 40%, about 50%, about 60%, about 70%, about 80%, about 90%, etc. of the distance 1430. The distance 1415 may be within a range from about 20% to about 80% of the distance 1430. The distance 1415 may be about 10%, about 20%, about 30%, about 40%, about 50%, about 60%, about 70%, about 80%, about 90%, etc. of the distance 1430. The distance 1417 may be within a range from about 20% to about 80% of the distance 1430. The distance 1417 may be about 10%, about 20%, about 30%, about 40%, about 50%, about 60%, about 70%, about 80%, about 90%, etc. of the distance 1430.

FIG. 14F illustrates a front exterior view of the distal end of seal 1400. In the illustrated example, the width of the seal may be equal to the diameter 1409 of the most proximal compressible member 1408. The width of the seal may be within an range from about 3 mm to about 40 mm, from about 3 mm to about 25 mm, from about 5 mm to about 20 mm, etc. The width of the seal may be greater than about 2 mm, about 3 mm, about 4 mm, about 5 mm, or more. The width of the seal may be less than about 40 mm, about 30 mm, about 20 mm, or less. The width of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 14F, the plurality of compressible members 1402, 1404, 1406, and 1408 may have a radially increasing exterior diameters from 1402 to 1408. While in some cases the diameters of the compressible members may not me monotonically increasing from 1402 to 1408, the diameters of the compressible members in the illustrated examples are monotonically increasing from 1402 to 1408.

The compressible members may have diameters 1403, 1405, 1407, and 1409. The diameter 1403 may be the diameter of the most distal compressible member 1402. The diameter 1409 may be the diameter of the most proximal compressible member 1408. The diameter 1405 may be the diameter of the more distal middle compressible member 1404. The diameter 1407 may be the diameter of the more proximal middle compressible member 1406. The diameter 1403 may be within a range from about 20% to about 80% of the diameter 1409. The diameter 1403 may be about 10%, about 20%, about 30%, about 40%, about 50%, about 60%, about 70%, about 80%, about 90%, etc. of the diameter 1409. The diameter 1405 may be within a range from about 20% to about 80% of the diameter 1409. The diameter 1405 may be about 10%, about 20%, about 30%, about 40%, about 50%, about 60%, about 70%, about 80%, about 90%, etc. of the diameter 1409. The diameter 1407 may be within a range from about 20% to about 80% of the diameter 1409. The diameter 1407 may be about 10%, about 20%, about 30%, about 40%, about 50%, about 60%, about 70%, about 80%, about 90%, etc. of the diameter 1409.

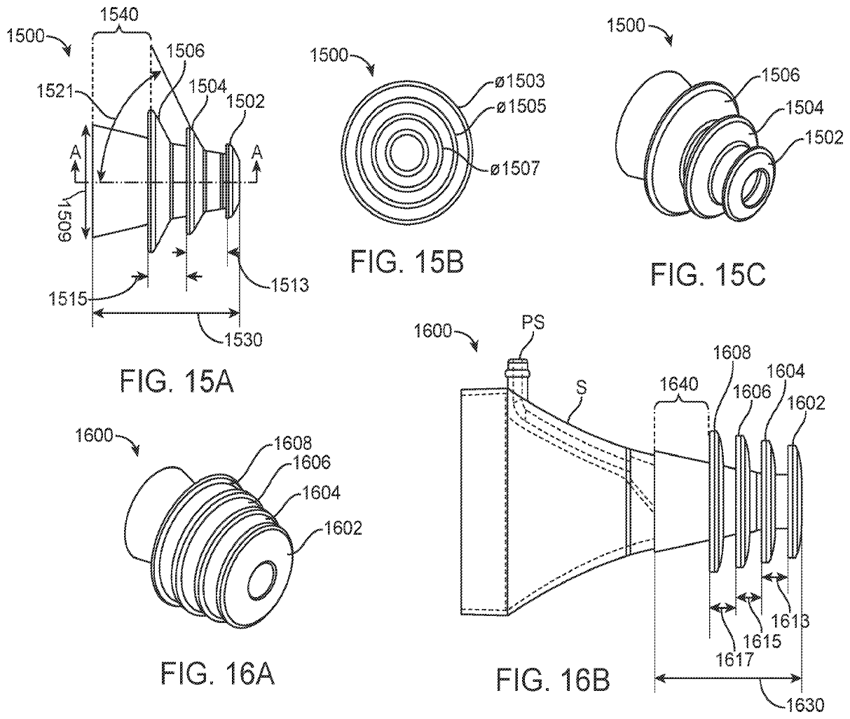

FIGS. 15A-15C illustrate an example of a seal 1500 with three flanges each comprising a cone shaped profile and a collar 1540. FIG. 15A illustrates a side exterior view of seal 1500. FIG. 15B illustrates a front view of a seal 1500. FIG. 15C illustrates a side isometric view of an exterior of a seal 1500. Seal 1500 may be coupled to an exterior of a speculum comprising a pneumatic source in a similar fashion to seal 1100 as disclosed herein.

Seal 1500 may comprise a setback (not shown) from a most distal edge of the speculum. A setback may be characterized as a distance from a most distal of the one or more compressible members to a most distal edge of the speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

As shown in FIG. 15A, the seal 1500 may also comprise collar 1540. Collar 1540 may comprise a cone configured to slide over a surface of a speculum. In some cases, a seal 1500 may be releasably coupled to a speculum. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1540 may comprise a length. In some cases, the length of collar 1540 may be greater than about 1 mm, about 2 mm, about 3 mm, about 4 mm, about 5 mm, about 6 mm, about 7 mm, about 8 mm, about 9 mm, or more. In some cases, the length of collar 1540 may be less than about 20 mm, about 19 mm, about 18 mm, about 17 mm, about 16 mm, about 15 mm or less. In some cases, the length of collar 1540 may be about 1 mm, about 2 mm, about 3 mm, about 4 mm, about 5 mm, about 6 mm, about 7 mm, about 8 mm, about 9 mm, etc. In some cases, the length of collar 1540 may be within a range from about 20 mm to about 1 mm, about 5 mm to about 15 mm, about 5 mm to about 7 mm, about 2 mm to about 5 mm, etc.

As shown in FIG. 15A, the seal 1500 may comprise a total length 1530. A total length 1530 may be greater than about 1 mm, about 2 mm, about 3 mm, about 4 mm, about 5 mm, about 6 mm, about 7 mm, about 8 mm, about 9 mm, or more. In some cases, a total length 1530 may be less than about 20 mm, about 19 mm, about 18 mm, about 17 mm, about 16 mm, about 15 mm or less. In some cases, the total length 1530 is about 1 mm, about 2 mm, about 3 mm, about 4 mm, about 5 mm, about 6 mm, about 7 mm, about 8 mm, about 9 mm, etc. A total length 1530 may be within a range from about 20 mm to about 1 mm, about 5 mm to about 15 mm, about 5 mm to about 7 mm, about 2 mm to about 5 mm, etc.

FIG. 15A illustrates an axis of symmetry A-A. In some cases, the seal may be radially symmetric about an axis. In some cases, a seal may not be radially symmetric.

The seal 1500 may comprise one or more compressible members 1502, 1504, and 1506. The one or more compressible members of seal 1500 may comprise flanges. In the example shown in FIG. 15A-15C, the seal may comprise 3 flanges; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more flanges. As illustrated, each of the flanges of seal 1500 may comprise a conical geometry.

As shown in FIG. 15A, the one or more compressible members may be spaced by distances 1513 and 1515. Distance 1513 may be a distance between compressible members 1502 and 1504. Distance 1515 may be a distance between compressible members 1504 and 1506. The distance 1513 may be within a range from about 20% to about 80% of the distance 1530. The distance 1513 may be about 10%, about 20%, about 30%, about 40%, about 50%, about 60%, about 70%, about 80%, about 90%, etc. of the distance 1530. The distance 1515 may be within a range from about 20% to about 80% of the distance 1530. The distance 1515 may be about 10%, about 20%, about 30%, about 40%, about 50%, about 60%, about 70%, about 80%, about 90%, etc. of the distance 1530.

FIG. 15B illustrates a front exterior view of the distal end of seal 1500. In the illustrated example, the width of the seal 1509 may be equal to the diameter 1507 of the compressible member 1506. The width 1509 of the seal may be within an range from about 3 mm to about 40 mm, from about 3 mm to about 25 mm, from about 5 mm to about 20 mm, etc. The width 1509 of the seal may be greater than about 2 mm, about 3 mm, about 4 mm, about 5 mm, or more. The width of the seal may be less than about 40 mm, about 30 mm, about 20 mm, or less. The width 1509 of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 15B, the plurality of compressible members 1502, 1504, 1506 may have a radially increasing exterior diameters from 1502 to 1506. While in some cases the diameters of the compressible members may not me monotonically increasing from 1502 to 1506, the diameters of the compressible members in the illustrated examples are monotonically increasing from 1502 to 1506.

The compressible members may have diameters 1503, 1505, and 1507. The diameter 1503 may be the diameter of the most distal compressible member 1502. The diameter 1507 may be the diameter of the most proximal compressible member 1506. The diameter 1505 may be the diameter of the middle compressible member 1504. The diameter 1503 may be within a range from about 20% to about 80% of the diameter 1507. The diameter 1503 may be about 10%, about 20%, about 30%, about 40%, about 50%, about 60%, about 70%, about 80%, about 90%, etc. of the diameter 1507. The diameter 1505 may be within a range from about 20% to about 80% of the diameter 1507. The diameter 1505 may be about 10%, about 20%, about 30%, about 40%, about 50%, about 60%, about 70%, about 80%, about 90%, etc. of the diameter 1507.

FIGS. 16A and 16B illustrate an example of a seal with four flanges each comprising a cone shaped profile. FIG. 16B illustrates an exterior isometric view of an exterior of speculum S comprising a seal 1600. FIG. 16A illustrates an angled isometric view of an exterior of seal 1600.

As shown in FIG. 16B, the speculum S may comprise a pneumatic source which may be fluidically coupled to an interior volume of the speculum, e.g. a lumen. The pressure source may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

In the illustrated embodiment, the seal 1600 is flush to the tip of the speculum. However, seals with similar geometries with any setback as disclosed herein may be possible.

The seal 1600 may also comprise collar 1640. Collar 1640 may comprise a cone configured to slide over a surface of a speculum. In some cases, a seal 1600 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1640 may comprise a length. In some cases, the length 1640 may be greater than about 1 mm, about 2 mm, about 3 mm, about 4 mm, about 5 mm, about 6 mm, about 7 mm, about 8 mm, about 9 mm, or more. In some cases, the length 1640 may be less than about 20 mm, about 19 mm, about 18 mm, about 17 mm, about 16 mm, about 15 mm or less. In some cases, the length 1640 may be about 1 mm, about 2 mm, about 3 mm, about 4 mm, about 5 mm, about 6 mm, about 7 mm, about 8 mm, about 9 mm, etc. In some cases, the length 1640 may be within a range from about 20 mm to about 1 mm, about 5 mm to about 15 mm, about 5 mm to about 7 mm, about 2 mm to about 5 mm, etc.

The seal of 1600 may comprise one or more compressible members 1602, 1604, 1606, and 1608. The one or more compressible members of seal 1600 may comprise flanges. In the example shown in FIG. 16A-16B, the seal may comprise 4 flanges; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more flanges.

Each of the flanges of seal 1600 comprises a similar geometry. Each compressible member may comprise a flat taper on the distal surface. Each compressible member may comprise a flat surface on the proximal surface.

As shown in FIG. 16B, the one or more compressible members may be spaced by distances 1613, 1615, and 1617. Distance 1613 may be a distance between compressible members 1602 and 1604. Distance 1615 may be a distance between compressible members 1604 and 1606. Distance 1617 may be a distance between compressible members 1606 and 1608. The distance 1613 may be within a range from about 20% to about 80% of the distance 1630. The distance 1613 may be about 10%, about 20%, about 30%, about 40%, about 50%, about 60%, about 70%, about 80%, about 90%, etc. of the distance 1630. The distance 1615 may be within a range from about 20% to about 80% of the distance 1630. The distance 1615 may be about 10%, about 20%, about 30%, about 40%, about 50%, about 60%, about 70%, about 80%, about 90%, etc. of the distance 1630. The distance 1617 may be within a range from about 20% to about 80% of the distance 1630. The distance 117 may be about 10%, about 20%, about 30%, about 40%, about 50%, about 60%, about 70%, about 80%, about 90%, etc. of the distance 1630.

As shown in FIG. 16B, the seal may comprise a total length 1630. The length of the seal may be within an range from about 3 mm to about 40 mm, from about 3 mm to about 25 mm, from about 5 mm to about 20 mm, etc. The length of the seal may be greater than about 2 mm, about 3 mm, about 4 mm, about 5 mm, or more. The length of the seal may be less than about 40 mm, about 30 mm, about 20 mm, or less. The length of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

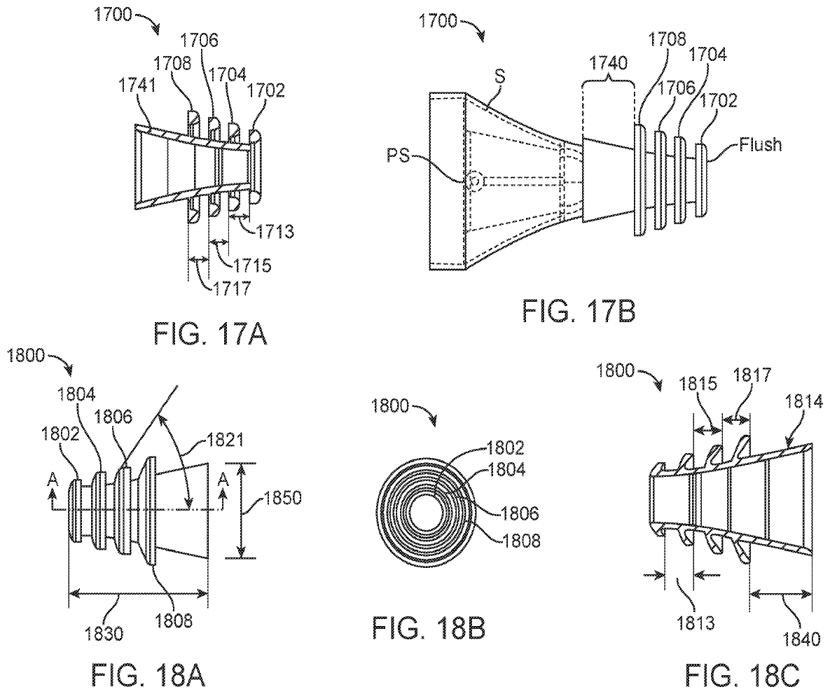

FIGS. 17A and 17B illustrate an example of a seal with 4 flanges in the form of flat tiers. FIG. 17A illustrates a section view of an interior a seal 1700. FIG. 17B illustrates an exterior isometric view of seal 1700 coupled to speculum S.

As shown in FIG. 17B, the speculum S may comprise a pneumatic source which may be fluidically coupled to an interior volume of the speculum, e.g. a lumen. The pressure source may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

In the illustrated embodiment, the seal 1700 is flush to the tip of the speculum. However, seals with similar geometries with any setback as disclosed herein may be possible.

The seal 1700 may also comprise collar 1740. Collar 1740 may comprise a cone configured to slide over a surface of a speculum. In some cases, a seal 1700 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1740 may comprise a length. In some cases, the length 1740 may be greater than about 1 mm, about 2 mm, about 3 mm, about 4 mm, about 5 mm, about 6 mm, about 7 mm, about 8 mm, about 9 mm, or more. In some cases, the length 1640 may be less than about 20 mm, about 19 mm, about 18 mm, about 17 mm, about 16 mm, about 15 mm or less. In some cases, the length 1740 may be about 1 mm, about 2 mm, about 3 mm, about 4 mm, about 5 mm, about 6 mm, about 7 mm, about 8 mm, about 9 mm, etc. In some cases, the length 1740 may be within a range from about 20 mm to about 1 mm, about 5 mm to about 15 mm, about 5 mm to about 7 mm, about 2 mm to about 5 mm, etc.

The seal of 1700 may comprise one or more compressible members 1702, 1704, 1706, and 1708. The one or more compressible members of seal 1700 may comprise flat tiers. In the example shown in FIG. 17A-17B, the seal may comprise 4 tiers; however, other numbers of tiers may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more tiers.

Each of the flanges of seal 1700 comprises a similar geometry. Each compressible member may comprise a flat surface the distal surface. Each compressible member may comprise a flat surface on the proximal surface.

As shown in FIG. 17A, the one or more compressible members may be spaced by distances 1713, 1715, and 1717. Distance 1713 may be a distance between compressible members 1702 and 1704. Distance 1715 may be a distance between compressible members 1704 and 1706. Distance 1717 may be a distance between compressible members 1706 and 1708. The distance 1713 may be within a range from about 20% to about 80% of the distance 1730. The distance 1713 may be about 10%, about 20%, about 30%, about 40%, about 50%, about 60%, about 70%, about 80%, about 90%, etc. of the distance 1730. The distance 1715 may be within a range from about 20% to about 80% of the distance 1730. The distance 1715 may be about 10%, about 20%, about 30%, about 40%, about 50%, about 60%, about 70%, about 80%, about 90%, etc. of the distance 1730. The distance 1717 may be within a range from about 20% to about 80% of the distance 1730. The distance 1717 may be about 10%, about 20%, about 30%, about 40%, about 50%, about 60%, about 70%, about 80%, about 90%, etc. of the distance 1730.

As shown in FIG. 17B, the seal may comprise a total length 1730. The length of the seal may be within an range from about 3 mm to about 40 mm, from about 3 mm to about 25 mm, from about 5 mm to about 20 mm, etc. The length of the seal may be greater than about 2 mm, about 3 mm, about 4 mm, about 5 mm, or more. The length of the seal may be less than about 40 mm, about 30 mm, about 20 mm, or less. The length of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 17B, a seal may comprise a constant wall thickness. In the illustrated embodiment a wall thickness at location 1741 is indicated. The wall thickness at location 1741 may be about 0.5 mm.

FIGS. 18A-18C illustrate an example of a seal with four flanges comprising space for a gel in a proximal volume. FIG. 18A illustrates a side exterior view of seal 1800. FIG. 18B illustrates a front view of a seal 1800. FIG. 18C illustrates a side section view of seal 1800.

Seal 1800 may comprise a setback (not shown) from a most distal edge of the speculum. A setback may be characterized as a distance from a most distal of the one or more compressible members to a most distal edge of the speculum. In some cases, seal 1800 is flush with a speculum. In some cases, a most distal of the one or more compressible members comprises a setback of greater than about 1 mm, about 2 mm, about 3 mm, about 4 mm, about 5 mm, about 6 mm, about 7 mm, about 8 mm, about 9 mm, or more. In some cases, a most distal of the one or more compressible members comprises a setback of less than about 20 mm, about 19 mm, about 18 mm, about 17 mm, about 16 mm, about 15 mm or less. In some cases, the setback is about 1 mm, about 2 mm, about 3 mm, about 4 mm, about 5 mm, about 6 mm, about 7 mm, about 8 mm, about 9 mm, etc. In the illustrated example, the setback may be about 2 mm. A setback may be within a range from about 20 mm to about 1 mm, about 5 mm to about 15 mm, about 5 mm to about 7 mm, about 2 mm to about 5 mm, etc.

As shown in FIG. 18B, the seal 1800 may also comprise collar 1840. Collar 1840 may comprise a cone configured to slide over a surface of a speculum. In some cases, a seal 1800 may be releasably coupled to a speculum. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1840 may comprise a length. In some cases, the length of collar 1840 may be greater than about 1 mm, about 2 mm, about 3 mm, about 4 mm, about 5 mm, about 6 mm, about 7 mm, about 8 mm, about 9 mm, or more. In some cases, the length of collar 1840 may be less than about 20 mm, about 19 mm, about 18 mm, about 17 mm, about 16 mm, about 15 mm or less. In some cases, the length of collar 1840 may be about 1 mm, about 2 mm, about 3 mm, about 4 mm, about 5 mm, about 6 mm, about 7 mm, about 8 mm, about 9 mm, etc. In some cases, the length of collar 1840 may be within a range from about 20 mm to about 1 mm, about 5 mm to about 15 mm, about 5 mm to about 7 mm, about 2 mm to about 5 mm, etc.

As shown in FIG. 18A, the seal 1800 may comprise a total length 1830. A total length 1830 may be greater than about 1 mm, about 2 mm, about 3 mm, about 4 mm, about 5 mm, about 6 mm, about 7 mm, about 8 mm, about 9 mm, or more. In some cases, a total length 1830 may be less than about 20 mm, about 19 mm, about 18 mm, about 17 mm, about 16 mm, about 15 mm or less. In some cases, the total length 1830 is about 1 mm, about 2 mm, about 3 mm, about 4 mm, about 5 mm, about 6 mm, about 7 mm, about 8 mm, about 9 mm, etc. A total length 1830 may be within a range from about 20 mm to about 1 mm, about 5 mm to about 15 mm, about 5 mm to about 7 mm, about 2 mm to about 5 mm, etc.

FIG. 18A illustrates an axis of symmetry A-A. In some cases, the seal may be radially symmetric about an axis. In some cases, a seal may not be radially symmetric.

The seal of 1800 may comprise one or more compressible members 1802, 1804, 1806, and 1808. The one or more compressible members of seal 1800 may comprise cones with space of gel in a proximal volume. In the example shown in FIG. 18A-18B, the seal may comprise 4 flanges; however, other numbers of flanges may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more flanges.

Each of the flanges of seal 1800 comprises a similar geometry. Each compressible member may comprise an angled surface the distal surface. Each compressible member may comprise an angled surface on the proximal surface. As shown the angle of the proximal surfaces may be slightly more acute than the distal angle. In some cases, the angle of the distal and proximal surfaces may be about equal. Angle 1821 of the distal surface may be less than about 90 degrees from the axis of symmetry A-A. Angle 1821 of the distal surface may be less than about 80 degrees, about 70 degrees, about 60 degrees, about 50 degrees, about 50 degrees, about 30 degrees, about 20 degrees or less from the axis of symmetry A-A, wherein the angle is measured in a proximal direction on axis A-A. Angle 1821 of the distal surface may be less than about 80 degrees, about 70 degrees, about 60 degrees, about 50 degrees, about 50 degrees, about 30 degrees, about 20 degrees or less from the axis of symmetry A-A, wherein the angle is measured in a distal direction on axis A-A.

The width of the seal may be equal to the diameter 1850 of the collar 1840. The width of the seal may be within an range from about 3 mm to about 40 mm, from about 3 mm to about 25 mm, from about 5 mm to about 20 mm, etc. The width of the seal may be greater than about 2 mm, about 3 mm, about 4 mm, about 5 mm, or more. The width of the seal may be less than about 40 mm, about 30 mm, about 20 mm, or less. The width of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

FIG. 18B illustrates a front view of the distal end of seal 1800. The plurality of compressible members 1802, 1804, 1806, and 1808 may have a radially increasing exterior diameters from 1802 to 1808. While in some cases the diameters of the compressible members may not me monotonically increasing from 1802 to 1808, the diameters of the compressible members in the illustrated examples are monotonically increasing from 1802 to 1808.

The compressible members may have diameters 1803, 1805, 1807, and 1809. The diameter 1803 may be the diameter of the most distal compressible member 1802. The diameter 1809 may be the diameter of the most proximal compressible member 1808. The diameter 1805 may be the diameter of the more distal middle compressible member 1804. The diameter 1807 may be the diameter of the more proximal middle compressible member 1806. The diameter 1803 may be within a range from about 20% to about 80% of the diameter 1809. The diameter 1803 may be about 10%, about 20%, about 30%, about 40%, about 50%, about 60%, about 70%, about 80%, about 90%, etc. of the diameter 1809. The diameter 1805 may be within a range from about 20% to about 80% of the diameter 1809. The diameter 1805 may be about 10%, about 20%, about 30%, about 40%, about 50%, about 60%, about 70%, about 80%, about 90%, etc. of the diameter 1809. The diameter 1807 may be within a range from about 20% to about 80% of the diameter 1809. The diameter 1807 may be about 10%, about 20%, about 30%, about 40%, about 50%, about 60%, about 70%, about 80%, about 90%, etc. of the diameter 1809.

As illustrated in FIG. 18C, the one or more compressible members may be spaced by distances 1813, 1815, and 1817. Distance 1813 may be a distance between compressible members 1802 and 1804. Distance 1815 may be a distance between compressible members 1804 and 1806. Distance 1817 may be a distance between compressible members 1806 and 1808. The distance 1813 may be within a range from about 20% to about 80% of the distance 1830. The distance 1813 may be about 10%, about 20%, about 30%, about 40%, about 50%, about 60%, about 70%, about 80%, about 90%, etc. of the distance 1830. The distance 1815 may be within a range from about 20% to about 80% of the distance 1830. The distance 1815 may be about 10%, about 20%, about 30%, about 40%, about 50%, about 60%, about 70%, about 80%, about 90%, etc. of the distance 1830. The distance 1817 may be within a range from about 20% to about 80% of the distance 1830. The distance 1817 may be about 10%, about 20%, about 30%, about 40%, about 50%, about 60%, about 70%, about 80%, about 90%, etc. of the distance 1830.

As shown in FIG. 18C, a seal may comprise a constant wall thickness. In the illustrated embodiment a wall thickness at location 1841 is indicated. The wall thickness at location 1841 may be about 0.5 mm.

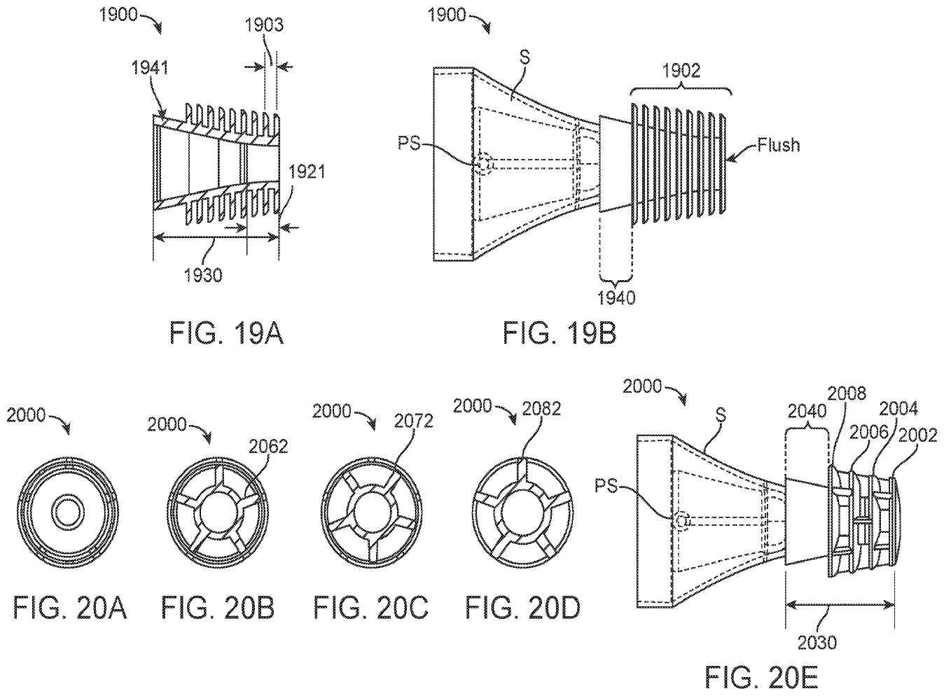

FIGS. 19A and 19B illustrate an example of a seal with 9 flanges in the form of flat tiers. FIG. 19A illustrates a section view of an interior a seal 1900. FIG. 19B illustrates an exterior isometric view of seal 1900 coupled to speculum S.

As shown in FIG. 19B, the speculum S may comprise a pneumatic source which may be fluidically coupled to an interior volume of the speculum, e.g. a lumen. The pressure source may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

In the illustrated embodiment, the seal 1900 is flush to the tip of the speculum. However, seals with similar geometries with any setback as disclosed herein may be possible.

The seal 1900 may also comprise collar 1940. Collar 1940 may comprise a cone configured to slide over a surface of a speculum. In some cases, a seal 1900 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 1940 may comprise a length. In some cases, the length 1940 may be greater than about 1 mm, about 2 mm, about 3 mm, about 4 mm, about 5 mm, about 6 mm, about 7 mm, about 8 mm, about 9 mm, or more. In some cases, the length 1640 may be less than about 20 mm, about 19 mm, about 18 mm, about 17 mm, about 16 mm, about 15 mm or less. In some cases, the length 1940 may be about 1 mm, about 2 mm, about 3 mm, about 4 mm, about 5 mm, about 6 mm, about 7 mm, about 8 mm, about 9 mm, etc. In some cases, the length 1940 may be within a range from about 20 mm to about 1 mm, about 5 mm to about 15 mm, about 5 mm to about 7 mm, about 2 mm to about 5 mm, etc.

The seal of 1900 may comprise one or more compressible members marked collectively as 1902. While the illustrated embodiment may have 9 compressible members other numbers may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more compressible members. The one or more compressible members of seal 1900 may comprise flat tiers; however, angled and varying shapes of the proximal and distal surfaces may be possible.

Each of the flanges of seal 1900 comprises a similar geometry. Each compressible member may comprise a flat surface the distal surface. Each compressible member may comprise a flat surface on the proximal surface.

As shown in FIG. 19A, the seal may comprise a total length 1930. The length of the seal may be within an range from about 3 mm to about 40 mm, from about 3 mm to about 25 mm, from about 5 mm to about 20 mm, etc. The length of the seal may be greater than about 2 mm, about 3 mm, about 4 mm, about 5 mm, or more. The length of the seal may be less than about 40 mm, about 30 mm, about 20 mm, or less. The length of the seal may be about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, etc.

As shown in FIG. 19A, the one or more compressible members may be spaced a distance 1903. In some cases, each of the compressible members is spaced by an equal distance as shown in FIG. 19A. In some cases, the distances may be increasing, may be decreasing or may be variably increasing and decreasing along the length of the seal. The distance 1803 may be within a range from about 20% to about 80% of the distance 1830. The distance 1803 may be about 10%, about 20%, about 30%, about 40%, about 50%, about 60%, about 70%, about 80%, about 90%, etc. of the distance 1830.

As shown in FIG. 19A, a seal may comprise a constant wall thickness. In the illustrated embodiment a wall thickness at location 1941 is indicated. The wall thickness at location 1941 may be about 0.5 mm.

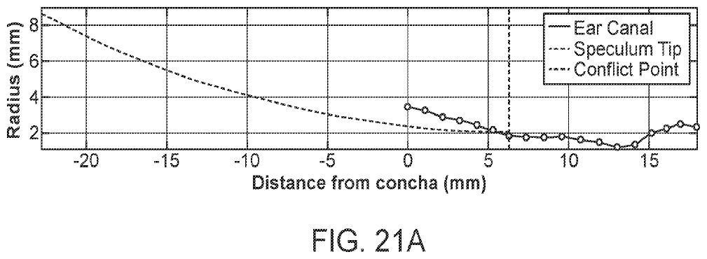

FIGS. 20A-20E illustrate an example of a seal formed with 4 flanges supported by perpendicular struts.

FIG. 20E illustrates an exterior isometric view of an exterior of speculum S comprising a seal 2000.

As shown in FIG. 20E, the speculum S may comprise a pneumatic source which may be fluidically coupled to an interior volume of the speculum, e.g. a lumen. The pressure source may be coupled to a pressure generator. The pressure generator may be disposed within a body of an otoscope. A pressure generator may generator a pressure challenge wave. A pressure challenge wave may comprise an air puff. The pressure challenge from the pressure source may propagate through the lumen. The pressure challenge may be a negative pressure change, a positive pressure change, or both as described elsewhere herein.

In the illustrated embodiment, the seal 2000 is flush to the tip of the speculum. However, seals with similar geometries with any setback as disclosed herein may be possible.

The seal 2000 may also comprise collar 2040. Collar 2040 may comprise a cone configured to slide over a surface of a speculum. In some cases, a seal 2000 may be releasably coupled to a speculum S. A seal which is releasably coupled may be removed from a speculum substantially without damage to the seal or the speculum. A seal which is substantially without damage may utilized to provide a sufficient seal for a pneumatic otoscopy measurement as disclosed elsewhere herein. In some cases, a seal may at least partly expand or stretch to fit onto a speculum and may compress (e.g., elastically) to be retained on the housing of the speculum. A seal may in some cases be permanently attached to a housing of the speculum. For example, a seal may be fused to the speculum, may be glued to the speculum, may be heat shrunk to the speculum, may be epoxied to the speculum. In some cases, the seal and the speculum may be substantially formed together so as to form a single part.

Collar 2040 may comprise a length. In some cases, the length 2040 may be greater than about 1 mm, about 2 mm, about 3 mm, about 4 mm, about 5 mm, about 6 mm, about 7 mm, about 8 mm, about 9 mm, or more. In some cases, the length 2040 may be less than about 20 mm, about 19 mm, about 18 mm, about 17 mm, about 16 mm, about 15 mm or less. In some cases, the length 2040 may be about 1 mm, about 2 mm, about 3 mm, about 4 mm, about 5 mm, about 6 mm, about 7 mm, about 8 mm, about 9 mm, etc. In some cases, the length 2040 may be within a range from about 20 mm to about 1 mm, about 5 mm to about 15 mm, about 5 mm to about 7 mm, about 2 mm to about 5 mm, etc.

The seal of 2000 may comprise one or more compressible members 2002, 2004, 2006, and 2008. The one or more compressible members of seal 2000 may comprise flat tiers. In the example shown in FIG. 20A-20E, the seal may comprise 4 tiers; however, other numbers of tiers may be possible. For example, a seal may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more tiers.

FIG. 20A shows a front view of a distal end of seal 2000. FIG. 20B shows a front section view of seal 2000 along an axis perpendicular to the axis of the lumen between compressible members 2002 and 2004. FIG. 20B shows struts 2062 between compressible members 2002 and 2004. While five struts are shown, other numbers of struts may be possible. FIG. 20C shows a front section view of seal 2000 along an axis perpendicular to the axis of the lumen between compressible members 2004 and 2006. FIG. 20C shows struts 2072 between compressible members 2004 and 2006. While five struts are shown, other numbers of struts may be possible. FIG. 20D shows a front section view of seal 2000 along an axis perpendicular to the axis of the lumen between compressible members 2006 and 2008. FIG. 20D shows struts 2082 between compressible members 2006 and 2008. While five struts are shown, other numbers of struts may be possible. For example, each tier may comprise 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, or more struts. As shown, each set of struts is radially displaced. The radial displacement may provide more even radial support around the axis of the lumen. In some cases, the struts may be radially aligned.

### Examples

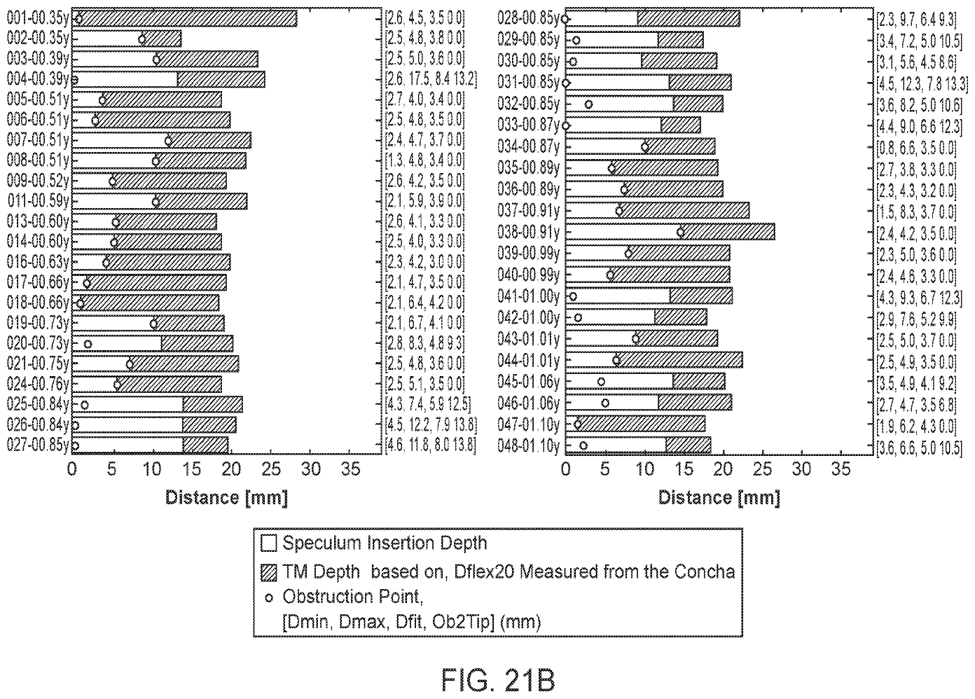

FIG. 21A is a plot of an ear insertion model for an example speculum tip inside of an example EAM geometry. The plot also indicates a conflict point between the speculum tip and the EAM.

FIG. 21B is a series of histograms for a plurality of subjects indicating: a length of the EAM, for example, the depth to the tympanic membrane; the modeled insertion depth; and the conflict point. The plot also shows a bracket indicating, in order, Dmin, Dmax, Dfit, and the distance from the obstruction point to the tip of the speculum. The variables Dmin, Dmax, and Dfit correspond to the diameters indicated in FIG. 3B. The diameter Dmin is the diameter of a circle defined by the circle which contacts the walls of the EAM at their nearest points when the EAM is at its narrowest. The diameter Dflex is defined to be 20% larger than Dmin. Dflex may allow for deformability of the EAM, which may enlarge to adapt to insertion of a speculum. The percent increase in diameter of 20% is an empirical parameter. Dflex may allow for better prediction of the contact point. Accordingly, the marked point in the histograms is the point of contact of the example speculum allowing a 20% flex. The diameter Dmax is the diameter of a circle defined by the circle which contacts the walls of the EAM at their nearest points when the EAM is at its widest. The subjected are indexed by the name {number-age}.

Based on user (e.g., clinician) feedback an insertion depth of about 3 to about 6 mm is useful to allow for imaging of a tympanic membrane.

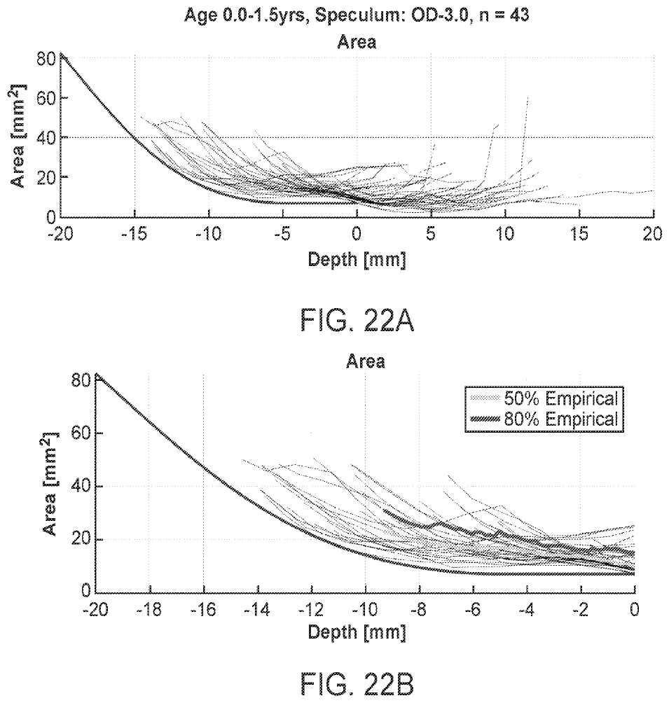

FIG. 22A is a plot including a series of EAM profiles aligned by conflict point with an example speculum tip. The plot comprises data from 43 subjects from age 0.0 to 1.5 years old. The example speculum comprises a 3.0 mm outer diameter with an extended constant diameter portion. The vertical axis indicates the cross-sectional area in square millimeters of the EAM as a function of depth. As shown, the speculum fits inside of the EAM's prior to the obstruction point.

FIG. 22B further illustrates the graph shown by FIG. 22A by showing only data proximal of the conflict point as well as the profile at which 50% and 80% of EAMs are sealed. The vertical axis indicates the cross-sectional area in square millimeters of the EAM as a function of depth. As shown, a speculum with a sufficient insertion depth may comprise a significant open cross-sectional area. In some cases, the 80% profile may comprise an experimentally generated profile of a seal of speculum. In some cases, the 80% profile maybe an approximate shape of a seal of speculum.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-80. (canceled)

81. A speculum operable to be disposed within an ear of a subject, the speculum comprising:
a housing comprising a lumen configured to allow a pressure challenge wave to be propagated therethrough, wherein the housing comprises a distal end sized and shaped to be disposed within an external auditory meatus of a subject; and
a seal comprising a surface sized and shaped to interface with the external auditory meatus, wherein the seal comprises a plurality of compressible flanges deformable to interface with the external auditory meatus, wherein a flange of the plurality of flanges comprises a gel within a volume radially near the housing, and wherein the seal is configured to retain a volume of fluid within the lumen in response to one or more of a negative pressure change or a positive pressure change.

82. The speculum of claim 81, wherein the seal is constructed from a material comprising a Shore durometer between about 40 to about 80 Shore-alpha.

83. The speculum of claim 81, wherein the plurality of compressible flanges comprises monotonically decreasing diameters.

84. The speculum of claim 81, wherein the volume is disposed on a proximal side of the flange.

85. The speculum of claim 81, wherein the volume is disposed on a distal side of the flange.

86. The speculum of claim 81, wherein the speculum is releasably couplable to a pneumatic otoscope.

87. The speculum of claim 86, wherein the pneumatic otoscope comprises an ultrasound transducer.

88. The speculum of claim 86, wherein the pneumatic otoscope comprises an indication that the pressure challenge wave within the lumen of the speculum is substantially retained within the lumen.

89. The speculum of claim 81, wherein a most distal of the one or more compressible members comprises a setback of greater than about 2 mm.

90. The speculum of claim 81, wherein a most distal of the one or more compressible members is configured to allow the speculum to enter at least about 5 mm within the external auditory meatus.

91. A seal for use with a speculum of an otoscope comprising:
a surface sized and shaped to interface with an external auditory meatus, wherein the seal comprises a plurality of compressible flanges deformable to interface with the external auditory meatus, wherein a flange of the plurality of flanges comprises a gel within a volume radially near the housing, and wherein the seal is configured to retain a volume of fluid within a lumen of the speculum in response to one or more of a negative pressure change or a positive pressure change.

92. The seal of claim 91, wherein the seal is constructed from a material comprising a Shore durometer between about 40 to about 80 Shore-alpha.

93. The seal of claim 91, wherein the plurality of compressible flanges comprises monotonically decreasing diameters.

94. The seal of claim 91, wherein the volume is disposed on a proximal side of the flange.

95. The seal of claim 91, wherein the volume is disposed on a distal side of the flange.

96. The seal of claim 91, wherein the seal is releasably couplable to a speculum of a pneumatic otoscope.

97. The seal of claim 96, wherein the pneumatic otoscope comprises an ultrasound transducer.

98. The seal of claim 96, wherein the pneumatic otoscope comprises an indication that the pressure challenge wave within the lumen of the speculum is substantially retained within the lumen.

99. The seal of claim 91, wherein a most distal of the one or more compressible members comprises a setback of greater than about 2 mm.

100. The seal of claim 91, wherein a most distal of the one or more compressible members is configured to allow the speculum to enter at least about 5 mm within the external auditory meatus.
